(19) United States
(12) Patent Application Publication
(10) Pub. No.: US 2006/0281714 A1
(43) Pub. Date: Dec. 14, 2006

(54) Combinations of a cathepsin k inhibitor and a bisphosphonate in the treatment of bone metastasis, tumor growth and tumor-induced bone loss

(76) Inventors: Johann Zimmermann, Auggen (DE); Carsten Goessl, Simi Valley, CA (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/565,453

(22) PCT Filed: Jul. 20, 2004
(86) PCT No.: PCT/EP04/08107

§ 371 Date: May 18, 2006

Related U.S. Application Data
(60) Provisional application No. 60/488,925, filed on Jul. 21, 2003.

(51) Int. Cl. A61K 31/675; A61K 31/66

(52) U.S. Cl. 514/89; 514/102

(57) ABSTRACT

This invention relates to pharmaceutical preparations comprising certain types of bisphosphonates and certain types of Cathepsin K inhibitors, in particular in the prevention and treatment of bone metastases, tumor-induced hypercalcemia, tumor growth, tumor-induced bone loss and bone loss diseases such as osteoporosis or cancer-therapy-induced bone loss

This invention relates to pharmaceutical preparations comprising certain types of bisphosphonates and certain types of Cathepsin K inhibitors, in particular in the prevention and treatment of bone metastases, tumor-induced hypercalcemia, tumor growth, tumor-induced bone loss and bone loss diseases such as osteoporosis or cancer-therapy-induced bone loss (e.g. by chemotherapy regimens, post bone marrow transplantation bone, sex-hormone depleting agents, such as bilateral orchiectomy/oophorectomy (surgical castration), LHRH analoga for premenopausal breast cancer or prostate cancer (chemical castration), chemotherapy inducing menopause in women with breast cancer, addition of aromatase inhibitors (e.g., letrozole) to LHRH therapy in premenopausal women with breast cancer or as stand-alone in postmenopausal women with breast cancer. In these cases the intention is to lower the already low sex-hormone levels to near undetectable levels).

Bisphosphonates are widely used to inhibit osteoclast activity in a variety of both benign and malignant diseases which involve excessive bone resorption. These pyrophosphate analogs not only reduce the occurrence of skeletal related events (e.g., fractures, need for radiation therapy, spinal cord compression, hypercalcemia of malignancy) but they also provide patients with further clinical benefit (e.g., pain reduction) and potentially improve survival. Bisphosphonates are able to prevent bone resorption in vivo; the therapeutic efficacy of bisphosphonates has been demonstrated in the treatment of osteoporosis, osteopenia, Paget's disease of bone, tumor-induced hypercalcemia (TIH) and, more recently, bone metastases (BM) from solid tumors and bone lesions from multiple myeloma (MM). The mechanisms by which bisphosphonates inhibit bone resorption are still not completely understood and seem to vary according to the bisphosphonates studied. Bisphosphonates have been shown to bind strongly to the hydroxyapatite crystals of bone, to reduce bone turnover and resorption, to decrease the levels of hydroxyproline or alkaline phosphatase in the blood, and in addition to inhibit the formation, recruitment, activation and the activity of osteoclasts.

Cathepsin K (cat K; also known as cathepsin O and cathepsin O₂) was cloned and found to be specifically expressed in osteoclasts (Shi G.-P., et al., 1995, FEBS Lett. 357: 129-134; Inoka T., et al., 1995, Biochem. Biophys. Res. Commun. 206: 89-96; Li Y., et al., 1995, J. Bone Miner. Res. 10: 1197-1202; Bromme D. et al., 1995, Biol. Chem. Hoppe-Seyler 376: 379-384; Tezuka, K et al., 1994, J Biol Chem 269: 1106-1109). Concurrent to the cloning, the autosomal recessive disorder, pycnodysostosis, characterized by an osteopetrotic phenotype with a decrease in bone resorption, was mapped to mutations present in the cat K gene (e.g. Gelb, B. D. et al., 1996, Science 273: 1236-1238). Human type I collagen, the major collagen in bone is a good substrate for cathepsin K (see e.g. Kafienah, W. et al., 1998, Biochem J 331: 727-732). In vitro experiments using antisense oligonucleotides to cat K, have shown diminished bone resorption in vitro, which is probably due to a reduction in translation of cat K mRNA (see Inui, T. et al., 1997, J Biol Chem 272: 8109-8112). Also, certain selective peptide based inhibitors of cat K have been developed (see e.g. U.S. Pat. No. 6,353,017) which can reduce bone resorption. Thus, their use in various disorders related to increased bone resorption, including inflammation, rheumatoid arthritis, osteoarthritis, osteoporosis, tumors (especially tumor invasion and tumor metastasis), coronary disease, atherosclerosis, autoimmune diseases, respiratory diseases, infectious diseases and immunological mediated diseases (including transplant rejection) have been proposed in mammals, in particular humans (see Brubaker, K. D. et al., 2001, J Bone Miner Res 18: 222-230 and Stroup, G. B., et al., 2001, J Bone Miner Res 16: 1739-1746).

Combination therapy of certain types of bisphosphonates and certain types of cat K inhibitors may have a number of benefits including more effectively treating the underlying bone loss of conditions such as in the following benign diseases, examples of which are various disorders related to increased bone resorption, including inflammation, rheumatoid arthritis, osteoarthritis, osteoporosis, tumors (especially tumor invasion and tumor metastasis), coronary disease, atherosclerosis, autoimmune diseases, respiratory diseases, infectious diseases and immunological mediated diseases (including transplant rejection), Paget's disease, periprosthetic bone loss or including the prevention of subchondral bone loss, osteophyte formation, and ultimately joint deterioration and destruction; but also malignant diseases such as osteolysis, due to tumor metastasis, including bone lesions from multiple myeloma, and, hypercalcemia of malignancy, bone metastases, tumors and cancer therapy-induced bone loss such as from the treatment from sex-hormone depleting agents, corticosteroid-containing chemotherapy regimens, post bone marrow transplantation bone loss. In particular, combination therapy of certain types of bisphosphonates and certain types of cat K inhibitors may be useful in the treatment of bone metastases, tumors and tumor-induced bone loss.

Accordingly the present invention provides a pharmaceutical composition for treatment of the above-mentioned diseases which comprises in combination certain types of bisphosphonates as described below and certain types of cat K inhibitors as described below for simultaneous, sequential or separate use.

Further the invention provides the use of certain types of cat K inhibitors as described below for the preparation of a medicament, for use in combination with certain types of bisphosphonates as described below for treatment of diseases as described above.

In the alternative the invention provides use of certain types of bisphosphonates as described below for the preparation of a medicament for use in combination with certain types of cat K inhibitors as described below for treatment of diseases as described above.

In a further aspect the invention provides a method of treating a patient suffering from a disease as described above comprising administering to the patient an effective amount of certain types of bisphosphonates as described below and an effective amount of certain types of cat K inhibitors as described below.

Yet further the invention provides use of certain types of cat K inhibitors as described below in combination with certain types of bisphosphonates to inhibit bone metastasis, inhibit cancer cell growth, inducing cancer cell apoptosis or/and inhibit cancer-therapy induced bone loss.

Accordingly also the present invention further provides a pharmaceutical composition for inhibiting bone metastasis, inhibiting cancer cell growth, inducing cancer cell apoptosis or/and inhibiting tumor-induced bone loss which comprises in combination certain types of cat K inhibitors as described below in combination with certain types of bisphosphonates for simultaneous, sequential or separate use.

Further the invention provides the use of certain types of bisphosphonates as described below for the preparation of a medicament, for use in combination with certain types of cat K inhibitors as described below for inhibiting bone metastasis, inhibiting cancer cell growth, inducing cancer cell apoptosis or/and inhibiting tumor-induced bone loss.

In a further aspect the invention provides a method of treating a patient suffering from bone metastasis, cancer cell growth, limited cancer cell apoptosis or/and cancer therapy-induced bone loss comprising administering to the patient an effective amount of certain types of bisphosphonates as described below and an effective amount of certain types of cat K inhibitors as described below.

In the present description the term “treatment” includes both prophylactic or preventative treatment as well as curative or disease modifying treatment, including treatment of patients at risk of contracting the disease or suspected to have contracted the disease as well as ill patients.

The invention is generally applicable to the treatment of malignant and benign diseases for which bisphosphonate and/or cat K inhibitor treatment is indicated or known to be effective. Preferably, the invention is applicable for malignant diseases, e.g. diseases with bone metastasis, diseases with tumor growth and tumor-induced bone loss (osteolysis). Examples of such diseases include cancers, such as breast and prostate cancers, multiple myeloma (MM), tumor induced hypertension (TIH) and similar diseases and conditions. In particular the invention is applicable to the treatment of multiple myeloma and bone metastases (BM) associated with cancers such as breast cancer, lung cancer, colon cancer, renal cancer or prostate cancer and other solid tumor cancers.

The compositions, uses and methods of the present invention represent an improvement to existing therapy of benign and/or malignant diseases in which bisphosphonates and/or cat K inhibitors are used. The combination of certain types of bisphosphonate as described below with certain types of cat K inhibitors as described below, advantageously gives rise to reduced levels of anti-metastatic, anti-tumorogenic and tumor-induced anti-osteolytic activity.

The bisphosphonates for use in the present invention are preferably N-bisphosphonates.

For the purposes of the present description an N-bisphosphonate is a compound which in addition to the characteristic geminal bisphosphate moiety comprises a nitrogen containing side chain, e.g. a compound of formula I 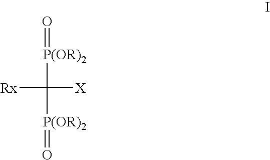
wherein
X is hydrogen, hydroxyl, amino, alkanoyl, or an amino group substituted by C₁-C₄alkyl, or alkanoyl;
R is hydrogen or C₁-C₄ alkyl and
Rx is a side chain which contains an optionally substituted amino group, or a nitrogen containing heterocycle (including aromatic nitrogen-containing heterocycles),
and pharmaceutically acceptable salts thereof or any hydrate thereof.

Thus, for example, suitable N-bisphosphonates for use in the invention may include the following compounds or a pharmaceutically acceptable salt thereof, or any hydrate thereof: 3-amino-1-hydroxypropane-1,1-diphosphonic acid (pamidronic acid), e.g. pamidronate (APD); 3-(N,N-dimethylamino)-1-hydroxypropane-1,1-diphosphonic acid, e.g. dimethyl-APD; 4-amino-1-hydroxybutane-1,1-diphosphonic acid (alendronic acid), e.g. alendronate; 1-hydroxy-3-(methylpentylamino)-propylidene-bisphosphonic acid, ibandronic acid, e.g. ibandronate; 6-amino-1-hydroxyhexane-1,1-diphosphonic acid, e.g. amino-hexyl-BP; 3-(N-methyl-N-n-pentylamino)-1-hydroxypropane-1,1-diphosphonic acid, e.g. methyl-pentyl-APD (=BM 21.0955); 1-hydroxy-2-(imidazol-1-yl)ethane-1,1-diphosphonic acid, e.g. zoledronic acid; 1-hydroxy-2-(3-pyridyl)ethane-1,1-diphosphonic acid (risedronic acid), e.g. risedronate, including N-methylpyridinium salts thereof, for example N-methylpyridinium iodides such as NE-10244 or NE-10446; 3-[N-(2-phenylthioethyl)-N-methylamino]-1-hydroxypropane-1,1-phosphonic acid; 1-hydroxy-3-(pyrrolidin-1-yl)propane-1,1-diphosphonic acid, e.g. EB 1053 (Leo); 1-(N-phenylaminothiocarbonyl)methane-1,1-phosphonic acid, e.g. FR 78844 (Fujisawa); 5-benzoyl-3,4-dihydro-2H-pyrazole-3,3-diphosphonic acid tetraethyl ester, e.g. U-81581 (Upjohn); and 1-hydroxy-2-(imidazo[1,2-a]pyridin-3-yl)ethane-1,1-diphosphonic acid, e.g. YM 529.

In one embodiment a particularly preferred N-bisphosphonate for use in the invention comprises a compound of Formula II 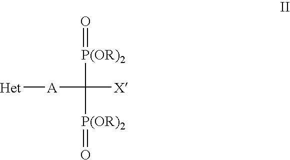
wherein
Het is an imidazole, oxazole, isoxazole, oxadiazole, thiazole, thiadiazole, pyridine, 1,2,3-triazole, 1,2,4-triazole or benzimidazole radical, which is optionally substituted by alkyl, alkoxy, halogen, hydroxyl, carboxyl, an amino group optionally substituted by alkyl or alkanoyl radicals or a benzyl radical optionally substituted by alkyl, nitro, amino or aminoalkyl;
A is a straight-chained or branched, saturated or unsaturated hydrocarbon moiety containing from 1 to 8 carbon atoms;
X′ is a hydrogen atom, optionally substituted by alkanoyl, or an amino group optionally substituted by alkyl or alkanoyl radicals, and
R is a hydrogen atom or an alkyl radical,
   and the pharmacologically acceptable salts thereof.

In a further embodiment a particularly preferred bisphosphonate for use in the invention comprises a compound of Formula III 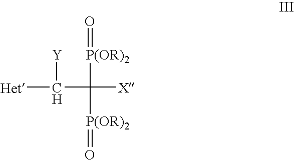
wherein
Het′ is a substituted or unsubstituted heteroaromatic five-membered ring selected from the group consisting of imidazolyl, imidazolinyl, isoxazolyl, oxazolyl, oxazolinyl, thiazolyl, thiazolinyl, triazolyl, oxadiazolyl and thiadiazolyl wherein said ring can be partly hydrogenated and wherein said substituents are selected from at least one of the group consisting of C₁-C₄ alkyl, C₁-C₄ alkoxy, phenyl, cyclohexyl, cyclohexylmethyl, halogen and amino and wherein two adjacent alkyl substituents of Het can together form a second ring;
Y is hydrogen or C₁-C₄ alkyl;
X″ is hydrogen, hydroxyl, amino, or an amino group substituted by C₁-C₄ alkyl, and R is hydrogen or C₁-C₄ alkyl;
   as well as the pharmacologically acceptable salts and isomers thereof.

In a yet further embodiment a particularly preferred bisphosphonate for use in the invention comprises a compound of Formula IV 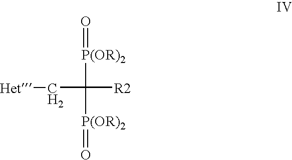
wherein
Het′″ is an imidazolyl, 2H-1,2,3-, 1H-1,2,4- or 4H-1,2,4-triazolyl, tetrazolyl, oxazolyl, isoxazolyl, oxadiazolyl, thiazolyl or thiadiazolyl radical which is unsubstituted or C-mono-or di-substituted by lower alkyl, by lower alkoxy, bx phenyl which may in turn be mono- or disubstituted by lower alkyl, lower alkoxy and/or halogen, by hydroxy, by di-lower alkylamino, by lower alkylthio and/or by halogen and is N-substituted at a substitutable N-atom by lower alkyl or by phenyl-lower alkyl which may in turn be mono- or di-substituted in the phenyl moiety by lower alkyl, lower alkoxy and/or halogen, and
R2 is hydrogen, hydroxy, amino, lower alkylthio or halogen,
lower radicals having up to and including 7 C-atoms,
   or a pharmacologically acceptable salt thereof.

Examples of particularly preferred N-bisphosphonates for use in the invention are:
2-(1-Methylimidazol-2-yl)-1-hydroxyethane-1,1-diphosphonic acid;
2-(1-Benzylimidazol-2-yl)-1-hydroxyethane-1,1-diphosphonic acid;
2-(1-Methylimidazol-4-yl)-1-hydroxyethane-1,1-diphosphonic acid;
1-Amino-2-(1-methylimidazol-4-yl)ethane-1,1-diphosphonic acid;
1-Amino-2-(1-benzylimidazol-4-yl)ethane-1,1-diphosphonic acid;
2-(1-Methylimidazol-2-yl)ethane-1,1-diphosphonic acid;
2-(1-Benzylimidazol-2-yl)ethane-1,1-diphosphonic acid;
2-(Imidazol-1-yl)-1-hydroxyethane-1,1-diphosphonic acid;
2-(imidazol-1-yl)ethane-1,1-diphosphonic acid;
2-4H-1,2,4-triazol-4-yl)-1-hydroxyethane-1,1-diphosphonic acid;
2-Thiazol-2-yl)ethane-1,1-diphosphonic acid;
2-(Imidazol-2-yl)ethane-1,1-diphosphonic acid;
2-(2-Methylimidazol-4-(5)-yl)ethane-1,1-diphosphonic acid;
2-(2-Phenylimidazol-4-(5)-yl)ethane-1,1-diphosphonic acid;
2-(4,5-Dimethylimidazol-1-yl)-1-hydroxyethane-1,1-diphosphonic acid, and
2-(2-Methylimidazol-4-(5)-yl)-1-hydroxyethane-1,1-diphosphonic acid,
   and pharmacologically acceptable salts thereof.

The most preferred N-bisphosphonate for use in the invention is 2-(imidazol-1yl)-1-hydroxyethane-1,1-diphosphonic acid (Zoledronic Acid) or a pharmacologically acceptable salt thereof, in particular Zoledronic Acid

All the N-bisphosphonic acid derivatives mentioned above are well known from the literature. This includes their manufacture (see e.g. EP-A-513760, pp. 13-48). For example, 3-amino-1-hydroxypropane-1,1-diphosphonic acid is prepared as described e.g. in U.S. Pat. No. 3,962,432 as well as the disodium salt as in U.S. Pat. Nos. 4,639,338 and 4,711,880, and 1-hydroxy-2-(imidazol-1-yl)ethane-1,1-diphosphonic acid is prepared as described e.g. in U.S. Pat. No. 4,939,130. See also U.S. Pat. Nos. 4,777,163 and 4,687,767.

The N-bisphosphonates may be used in the form of an isomer or of a mixture of isomers where appropriate, typically as optical isomers such as enantiomers or diastereoisomers or geometric isomers, typically cis-trans isomers. The optical isomers are obtained in the form of the pure antipodes and/or as racemates.

The N-bisphosphonates can also be used in the form of their hydrates or include other solvents used for their crystallisation.
The cat K inhibitors used in the pharmaceutical compositions and treatment methods of the present invention typically comprises a compound of formula V, or a physiologically acceptable and -cleavable ester or a salt thereof 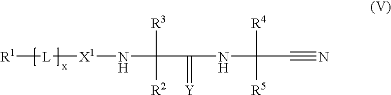
wherein R¹ is optionally substituted (aryl, aryl-lower alkyl, lower alkenyl, lower alkynyl, heterocyclyl or heterocyclyl-lower alkyl);
R² and R³ together represent lower alkylene, optionally interrupted by O, S or NR⁶, so as to form a ring with the carbon atom to which they are attached, and R⁶ is hydrogen, lower alkyl or aryl-lower alkyl;
R⁴ and R⁵ are independently H, or optionally substituted (lower alkyl or aryl-lower alkyl), —C(O)OR⁷, or —C(O)NR⁷R⁸, wherein R⁷ is optionally substituted (lower alkyl, aryl, aryl-lower alkyl, cycloalkyl, bicycloalkyl, bicycloalkyl or heterocyclyl), and R⁸ is H, or optionally substituted (lower alkyl, aryl, aryl-lower alkyl, cycloalkyl, bicycloalkyl, bicycloalkyl or heterocyclyl); or
R⁴ and R⁵ together represent lower alkylene, optionally interrupted by O, S or NR⁶, so as to form a ring with the carbon atom to which they are attached, and R⁶ is hydrogen, lower alkyl or aryl-lower alkyl; or
R⁴ is H or optionally substituted lower alkyl and R⁵ is a substituent of formula —X²—(Y¹)—(Ar)ₚ-Q-Z wherein
Y¹ is O, S, SO, SO₂, N(R⁶)SO₂, N—R⁶, SO₂NR⁶, CONR⁶ or NR⁶CO;
N is zero or one;
P is zero or one;
X² is lower alkylene: or when n is zero, X² is also C₂-C₇-alkylene interrupted by O, S, SO, SO₂, NR⁶, SO₂NR⁶, CONR⁶ or NR⁶CO, and R⁶ is hydrogen, lower alkyl or aryl-lower alkyl;
Ar is arylene;
Z is hydroxyl, acyloxy, carboxyl, esterified carboxyl, amidated carboxyl, aminosulfonyl, (lower alkyl or aryl-lower alkyl)aminosulfonyl, or (lower alkyl or aryl-lower alkyl)sufonylaminocarbonyl; or Z is tetrazolyl, triazolyl or imidazolyl;
Q is a direct bond, lower alkylene, Y¹-lower alkylene or C₂-C₇-alkylene interrupted by Y¹;
X¹ is —C(O)—, —C(S)—, —S(O)—, —S(O)₂—, or —P(O)(OR⁶)—, and R⁶ is as defined above;
Y is oxygen or sulphur;
L is optionally substituted -Het-, -Het-CH₂— or —CH₂-Het-, and Het is a hetero atom selected from O, N or S; and
X is zero or one; and
aryl in the above definitions represents carbocyclic or heterocyclic aryl.

Particular compounds of formula V are those wherein R¹ is a substituted phenyl, e.g. whereas the substituent is an optionally substituted nitrogen-containing heterocyclic substituent (=Het^{IV}). This substituent may be at the 2- or 3-position of the phenyl ring, though preferably at the 4-postion. Het^{IV} signifies a heterocyclic ring system containing at least one nitrogen atom, from 2 to 10, preferably from 3 to 7, most preferably 4 or 5, carbon atoms and optionally one or more additional heteroatoms selected from O, S or preferably N.

Het^{IV} may comprise an unsaturated, e.g. an aromatic, nitrogen-containing heterocycle; though preferably comprises a saturated nitrogen-containing heterocycle. Particularly preferred saturated nitrogen-containing heterocycles are piperazinyl, preferably piperazin-1-yl, or piperidinyl, preferably piperidinyl.

Het^{IV} may be substituted by one or more substituents, e.g. by up to 5 substituents independently selected from halogen, hydroxy, amino, nitro, optionally substituted C₁₋₄alkyl (e.g. alkyl substituted by hydroxy, alkyloxy, amino, optionally substituted alkylamino, optionally substituted dialkylamino, aryl or heterocyclyl), C₁₋₄alkoxy. Preferably Het^{IV} is substituted at a nitrogen atom, most preferably mono-substituted at a nitrogen atom. Preferred substituents for Het^{IV} are C₁-C₇lower alkyl, C₁-C₇lower alkoxy-C₁-C₇lower alkyl, C₅-C₁₀aryl-C₁-C₇lower alkyl, or C₃-C₈cycloalkyl.

Particularly preferred embodiments of the invention provides a compound of formula VI, or a pharmaceutically acceptable salt or ester thereof 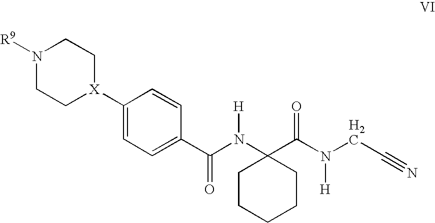
wherein X is CH or N, and
R⁹ is H, C₁-C₇lower alkyl, C₁-C₇lower alkoxy-C₁-C₇lower alkyl, C₅-C₁₀aryl-C₁-C₇lower alkyl, or C₃-C₈cycloalkyl.

Thus particular examples of R⁹ as C₁-C₇lower alkyl are methyl, ethyl, n-propyl, or i-propyl are preferred. A particular example of R as C₁-C₇lower alkoxy-C₁-C₇lower alkyl is methoxyethyl. A particular example of R as C₅-C₁₀aryl-C₁-C₇lower alkyl is benzyl. A particular example of R as C₃-C₈cycloalkyl is cyclopentyl. Examples of particular compounds of formula VI are: N-[1-(Cyanomethyl-carbamoyl)-cyclohexyl]-4-(piperazin-1-yl)-benzamide; N-[1-(Cyanomethyl-carbamoyl)-cyclohexyl]-4-(4-methyl-piperazin-1-yl)-benzamide; N-[1-(Cyanomethyl-carbamoyl)-cyclohexyl]-4-(4-ethyl-piperazin-1-yl)-benzamide; N-[1-Cyanomethyl-carbamoyl)-cyclohexyl]-4-[4-(1-propyl)-piperazin-1-yl]-benzamide; N-[1-(Cyanomethyl-carbamoyl)-cyclohexyl]4-(4-isopropyl-piperazin-1-yl)-benzamide; N-[1-(Cyanomethyl-carbamoyl)-cyclohexyl]4-(4-benzyl-piperazin-1-yl)-benzamide; N-[1-(Cyanomethyl-carbamoyl)-cyclohexyl]4-[4-(2-methoxy-ethyl)-piperazin-1-yl]-benzamide; N-[1-(Cyanomethyl-carbamoyl)-cyclohexyl]4-(1-propyl-piperidinyl)-benzamide; N-[1-(Cyanomethyl-carbamoyl)-cyclohexyl]-4-[1-(2-methoxy-ethyl)-piperidin-4-yl]-benzamide; N-[1-(Cyanomethyl-carbamoyl)-cyclohexyl](1-isopropyl-piperidin-4-yl)-benzamide; N-[1-(Cyanomethyl-carbamoyl)-cyclohexyl]-4-(1-cyclopentyl-piperidin-4-yl)-benzamide; N-[1-(Cyanomethyl-carbamoyl)-cyclohexyl]-4-(1-methyl-piperidin-4-yl)-benzamide, and N-[1-(Cyanomethyl-carbamoyl)-cyclohexyl]-4-(piperidin-4-yl)-benzamide.

The most preferred cat K inhibitor for use in the invention is N-[1-(Cyanomethyl-carbamoyl)-cyclohexyl]-4-[4-(1-propyl)-piperazin-1-yl]-benzamide or a pharmacologically acceptable salt thereof, e.g. hydrogen maleate salt thereof.

All the cat K inhibitors mentioned above are known from the literature. This includes their production (see e.g. U.S. Pat. No. 6,353,017B1, pp. 15-17).
An alternative class of cat K inhibitors compounds for use in the invention comprises a compound of formula VII, or a physiologically acceptable and -cleavable ester or a salt thereof 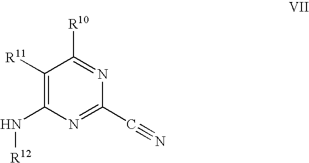
wherein
R¹⁰ is H, —R¹⁴, —OR¹⁴ or NR¹³R¹⁴,
wherein R¹³ is H, lower alkyl or C₃ to C₁₀ cycloalkyl, and
R¹⁴ is lower alkyl or C₃ to C₁₀ cycloalkyl, and
wherein R¹³ and R¹⁴ are independently, optionally substituted by halo, hydroxy, lower alkoxy, CN, NO₂, or optionally mono- or di-lower alkyl substituted amino;
R¹¹ is SO—NR¹⁵R¹⁶, —NH—CO—R¹⁵, —CH₂—NH—C(O)—R¹⁵, —CO—R¹⁵, —S(O)—R¹⁵, —S(O)₂—R⁵, —CH₂—CO—R¹⁵ or —CH₂—NR¹⁵R¹⁶,
wherein
R¹⁵ is aryl, aryl-lower alkyl, C₃-C₁₀cycloalkyl, C₃-C₁₀cycloalkyl-lower alkyl, heterocyclyl or heterocyclyl-lower alkyl,
R¹⁶ is H, aryl, aryl-lower alkyl, aryl-lower-alkenyl, C₃-C₁₀cycloalkyl, C₃-C₁₀cycloalkyl-lower alkyl, heterocyclyl or heterocyclyl-lower alkyl, or
wherein R¹⁵ and R¹⁶ together with the nitrogen atom to which they attached are joined to form an N-heterocyclyl group,
wherein N-heterocyclyl denotes a saturated, partially unsaturated or aromatic nitrogen containing heterocyclic moiety attached via a nitrogen atom thereof having from 3 to 8 ring atoms optionally containing a further 1, 2 or 3 heteroatoms selected from N, NR¹⁷, O, S, S(O) or S(O)₂ wherein R¹⁷ is H or optionally substituted (lower alkyl, carboxy, acyl (including both lower alkyl acyl, e.g. formyl, acetyl or propionyl, or aryl acyl, e.g. benzoyl), amido, aryl, S(O) or S(O)₂), and wherein the N-heterocyclyl is optionally fused in a bicyclic structure, e.g. with a benzene or pyridine ring, and wherein the N-heterocyclyl is optionally linked in a spiro structure with a 3 to 8 membered cycloalkyl or heterocyclic ring wherein the heterocyclic ring has from 3 to 10 ring members and contains from 1 to 3 heteroatoms selected from N, NR¹⁶, O, S, S(O) or S(O)₂ wherein R¹⁶ is as defined above), and wherein heterocyclyl denotes a ring having from 3 to 10 ring members and containing from 1 to 3 heteroatoms selected from N, NR¹⁷, O, S, S(O) or S(O)₂ wherein R¹⁷ is as defined above), and wherein R¹⁵ and R¹⁶ are independently, optionally substituted by one or more groups, e.g. 1-3 groups, selected from halo, hydroxy, oxo, lower alkoxy, CN or NO₂, or optionally substituted (optionally mono- or di-lower alkyl substituted amino, lower-alkoxy, aryl, aryl-lower alkyl, N-heterocyclyl or N-heterocyclyl-lower alkyl (wherein the optional substitution comprises from 1 to 3 substituents selected from halo, hydroxy, lower alkoxy, lower alkoxy-lower alkyl, lower alkoxy-carbonyl, CN, NO₂, N-heterocyclyl or N-heterocyclyl-lower alkyl, or optionally mono- or di-lower alkyl substituted amino; R¹² is is independently H, or optionally substituted (lower alkyl, aryl, aryl-lower alkyl, C₃-C₁₀cycloalkyl, C₃-C₁₀cycloalkyl-lower alkyl, heterocyclyl or heterocyclyl-lower alkyl), and wherein R2 is optionally substituted by halo, hydroxy, oxo, lower alkoxy, CN, NO₂, or optionally mono- or di-lower alkyl substituted amino.

Halo or halogen denote I, Br, Cl or F.

The term “lower” referred to above and hereinafter in connection with organic radicals or compounds respectively defines such as branched or unbranched with up to and including 7, preferably up to and including 5 and advantageously one, two or three carbon atoms. A lower alkyl group is branched or unbranched and contains 1 to 7 carbon atoms, preferably 1-5 carbon atoms. Lower alkyl represents; for example, methyl, ethyl, propyl, butyl, isopropyl isobutyl, tertiary butyl or neopentyl (2,2-dimethylpropyl).

Halo-substituted lower alkyl is C₁-C₇lower alkyl substituted by up to 6 halo atoms.

A lower alkoxy group is branched or unbranched and contains 1 to 7 carbon atoms, preferably 1-4 carbon atoms. Lower alkoxy represents for example methoxy, ethoxy, propoxy, butoxy, isopropoxy, isobutoxy or tertiary butoxy.

A lower alkene, alkenyl or alkenyloxy group is branched or unbranched and contains 2 to 7 carbon atoms, preferably 2-4 carbon atoms and contains at least one carbon-carbon double bond. Lower alkene lower alkenyl or lower alkenyloxy represents for example vinyl, prop-1-enyl, allyl, butenyl, isopropenyl or isobutenyl and the oxy equivalents thereof.

A lower alkyne, alkynyl or alkynyloxy group is branched or unbranched and contains 2 to 7 carbon atoms, preferably 2-4 carbon atoms and contains at least one carbon-carbon triple bond. Lower alkyne or alkynyl represents for example ethynyl, prop-1-ynyl, propargyl, butynyl, isopropynyl or isobutynyl and the oxy equivalents thereof.

In the present description, oxygen containing substituents, e.g. alkoxy, alkenyloxy, alkynyloxy, carbonyl, etc. encompass their sulphur containing homologues, e.g. thioalkoxy, thioalkenyloxy, thioalkynyloxy, thiocarbonyl, sulphone, sulphoxide etc.

Aryl represents carbocyclic or heterocyclic aryl.

Carbocyclic aryl represents monocyclic, bicyclic or tricyclic aryl, for example phenyl or phenyl mono-, di- or tri-substituted by one, two or three radicals selected from lower alkyl, lower alkoxy, aryl, hydroxy, halogen, cyano, trifluoromethyl, lower alkylenedioxy and oxy-C₂-C₃-alkylene and other substituents, for instance as described in the examples; or 1- or 2-naphthyl; or 1- or 2-phenanthrenyl. Lower alkylenedioxy is a divalent substituent attached to two adjacent carbon atoms of phenyl, e.g. methylenedioxy or ethylenedioxy. Oxy-C₂-C₃-alkylene is also a divalent substituent attached to two adjacent carbon atoms of phenyl, e.g. oxyethylene or oxypropylene. An example for oxy-C₂-C₃-alkylene-phenyl is 2,3-dihydrobenzofuran-5-yl.

Preferred as carbocyclic aryl is naphthyl, phenyl or phenyl optionally substituted, for instance, as described in the examples, e.g. mono- or disubstituted by lower alkoxy, phenyl, halogen, lower alkyl or trifluoromethyl.

Heterocyclic aryl represents monocyclic or bicyclic heteroaryl, for example pyridyl, indolyl, quinoxalinyl, quinolinyl, isoquinolinyl, benzothienyl, benzofuranyl, benzopyranyl, benzothiopyranyl, furanyl, pyrrolyl, thiazolyl, oxazolyl, isoxazolyl, triazolyl, tetrazolyl, pyrazolyl, imidazolyl, thienyl, or any said radical substituted, especially mono- or di-substituted as defined above.

Preferably, heterocyclic aryl is pyridyl, indolyl, quinolinyl, pyrrolyl, thiazolyl, isoxazolyl, triazolyl, tetrazolyl, pyrazolyl, imidazolyl, thienyl, or any said radical substituted, especially mono- or di-substituted as defined above.

Cycloalkyl represents a saturated cyclic hydrocarbon optionally substituted by lower alkyl which contains 3 to 10 ring carbons and is advantageously cyclopropyl, cyclopentyl, cyclohexyl, cycloheptyl or cyclooctyl optionally substituted by lower alkyl.

N-heterocyclyl is as defined above. Preferred N-heterocyclic substituents are optionally substituted pyrrolidine, pyrrole, diazole, triazole, tetrazole, imidazole, oxazole, thiazole, pyridine, pyrimidine, triazine, piperidine, piperazine, morpholine, phthalimde, hydantoin, oxazolidinone or 2,6-dioxo-piperazine and, for example, as hereinafter described in the examples.

In a further embodiment the invention provides a compound of formula VIII, or a pharmaceutically acceptable salt or ester thereof 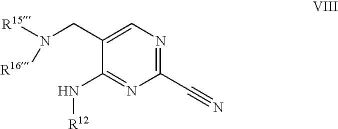
wherein R¹² is as defined above and R¹⁵′″ and R¹⁶′″ are as defined above for R¹⁵ and R¹⁶ respectively.

R¹² is preferably R¹²′ which is lower alkyl, e.g. straight chain or more preferably branched-chain C₁-C₆ alkyl, e.g. especially 2-ethylbutyl, isobutyl, or 2,2-dimethylpropyl; or C₃-C₆cycloalkyl, especially cyclopropyl, cyclopentyl or cyclohexyl.

R¹⁵′″ and R¹⁶′″ may be such that R¹⁵′″ and R¹⁶′″ together with the nitrogen atom to which they are joined to form an N-heterocyclyl group. R¹⁵′″ is preferably optionally substituted (aryl-lower-alkyl, heterocyclyl-aryl, N-heterocyclyl-aryl or aryl-N-heterocyclyl (where N-heterocyclyl is as defined above). R¹⁵′″ is preferably optionally substituted by from 1-4 substituents selected from halo, hydroxy, nitro, cyano, lower-alkyl, lower-alkoxy or lower-alkoxy-lower-akyl. For example, R¹⁵′″ is 4-methoxy-benzyl, 3-methoxy-benzyl, 4-(4-methyl-piperazin-1-yl)-benzyl, 4-[4-(2-ethoxy-ethyl)-piperazin-1-yl]-benzyl, 1-methyl-1-phenyl-ethyl, 2-(4-methoxy-phenyl)-1,1-dimethyl-ethyl, 2-(4-fluoro-phenyl)-1,1-dimethyl-ethyl, 4-(4-methyl-piperazin-1-yl)-phenyl]-ethyl, 2-[4-(4-isopropyl-piperazin-1-yl)-phenyl]-1,1-dimethyl-ethyl, 2-{4-[4-(2-methoxy-ethyl)-piperazin-1-yl]-phenyl)-1,1-dimethyl-ethyl, 2-{3-[4-(2-ethoxy-ethyl)-piperazin-1-yl]-phenyl}-1,1-dimethyl-ethyl, 2-[3-(4-ethyl-piperazin-1-yl)-phenyl]-1,1-dimethyl-ethyl, 2-[3-(4-isopropyl-piperazin-1-yl)-phenyl]-1,1-dimethyl-ethyl, 1,1-dimethyl-2-(3-pyrrolidin-1-yl-phenyl)-ethyl, 2-{3-[4-(2-methoxy-ethyl)-piperazin-1-yl]-phenyl}-1,1-dimethyl-ethyl, 2-(4-methoxy-phenyl)-ethyl, 2-[4-(4-methyl-piperazin-1-yl)-phenyl]-ethyl, 2-[4-(4-isopropyl-piperazin-1-yl)-phenyl]-ethyl, 2-{4-[4-(2-methoxy-ethyl)-piperazin-1-yl]-phenyl}-ethyl, 2-(3-methoxy-phenyl)-ethyl, 2-[3-(4-methyl-piperazin-1-yl)-phenyl]-ethyl, 2-[4-(4-isopropyl-piperazin-1-yl)-phenyl]-ethyl, 2-pyrrol-1-yl-ethyl, 3-piperidin-1-yl-propyl, 2-(4-methoxy-phenyl)-2-methyl-propyl, 2-methyl-2-[4-(4-methyl-piperazin-1-yl)-phenyl]-propyl, 2-[4-(4-isopropyl-piperazin-1-yl)-phenyl]-2-methyl-propyl, 2-{4-[4-(2-ethoxy-ethyl)-piperazin-1-yl]-phenyl}-2-methyl-propyl, 2-(4-[pyrimidin-1-yl]-phenyl}-2-methyl-propyl, 4-(3-methoxy-phenyl)-piperazin-1-yl-methyl, 4-(4-methoxy-phenyl)-piperazin-1-yl-methyl, 1-methyl-1-(1-phenyl-cyclopropyl)-ethyl. For example, R¹⁵′″ and R¹⁶′″ together with the nitrogen atom to which they are joined to form an N-heterocyclyl group is 4-(2-pyridinyl-ethyl)-piperazin-1-yl, [4-(2-pyridin-2-yl-ethyl)-piperazin-1-yl, 4-pyridin ylmethyl-piperazin-1-yl, 4-(2-piperidin-1-yl-ethyl)-piperazin-1-yl, 4-(2-pyrrolidin-1-yl-ethyl)-piperazin-1-yl, 4-(2-Diethylamino-ethyl)-piperazin-1-yl, 4-(3-Diethylamino-propyl)-piperazin-1-yl, 4-(1-methyl-piperidin-4-yl)-piperazin-1-yl, 4-pyrrolidin-1-yl-piperidin-1-yl, 4-(2-methoxy-ethyl)-piperazin-1-yl.

In a preferred embodiment the invention provides a compound of formula IX, or a pharmaceutically acceptable salt or ester thereof 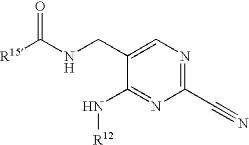
wherein R¹² is as defined above and R¹⁵′ is as defined above for R¹⁵.

R¹² is preferably R¹²′ which is lower alkyl, e.g. straight chain or more preferably branched-chain C₁-C₆ alkyl, e.g. especially 2-ethylbutyl, isobutyl, or 2,2-dimethylpropyl; or C₃-C₆cycloalkyl, especially cyclopropyl, cyclopentyl or cyclohexyl.

R¹⁵′ is preferably optionally substituted (aryl-lower-alkyl, heterocyclyl-aryl, N-heterocyclyl-aryl or aryl-N-heterocyclyl (where N-heterocyclyl is as defined above). R¹⁵′ is preferably optionally substituted by from 1-4 substituents selected from halo, hydroxy, nitro, cyano, lower-alkyl, lower-alkoxy, lower-alkoxy-carbonyl or lower-alkoxy-lower-akyl. For example, R¹⁵′ is 4-methoxy-phenyl, 4-(1-propyl-piperidin-4-yl)-phenyl, 4-(4-methyl-piperazin-1-yl)-phenyl, 4-[1-(2-methoxy-ethyl)-piperidin-4-yl]-phenyl, 4-(4-propyl-piperazin-1-yl)-phenyl, 3-[4-(4-methyl-piperazin-1-yl)-phenyl]-propionyl, 3-[3-(4-methyl-piperazin-1-yl)-phenyl]-propionyl, 4-(4-ethyl-piperazin-1-yl)-phenyl, 4-(4-isopropyl-piperazin-1-yl)-phenyl, 4-[4-(2-ethoxy-ethyl)-piperazin-1-yl]-phenyl, 4-[4-(2-methoxy-ethyl)-piperazin-1-yl]-phenyl, 4-piperazin-1-yl-phenyl, 4-[4-(carboxylic acid tert-butyl ester) piperazino-1-yl-]-phenyl, 3-[4-(carboxylic acid tert-butyl ester)piperazino-1-yl-]-phenyl, 3-(4-methyl-piperazin-1-yl)-phenyl, 3-(4-ethyl-piperazin-1-yl)-phenyl, 3-(4-isopropyl-piperazin-1-yl)-phenyl, 3-[4-(2-methoxy-ethyl)-piperazin-1-yl]-phenyl, 3-[4-(2-ethoxy-ethyl)-piperazin-1-yl]-phenyl, 3-(2-pyrrolidin-1-yl-ethoxy)-phenyl, 3-(2-dimethylamino-ethoxy)-4-methoxy-phenyl, 4-dimethylaminomethyl-phenyl, 4-(4-methyl-piperazin-1-ylmethyl)phenyl, 4-[1-(2-methoxy-ethyl) piperidin-4-ylmethyl]-phenyl, 4-methoxy-3-(2-piperidin-1-yl-ethoxy)-phenyl, 3-[4-ethyl-piperazin-1-yl)-phenyl]-2,2-dimethyl-propionyl, 3-[4-(4-propyl-piperazin-1-yl)-phenyl]-propionyl, 3-(4-pyrrolidin-1-yl-phenyl)-propionyl, 3-[3-(4-ethyl-piperazin-1-yl)-phenyl]-2,2-dimethyl-propionyl, 3-{3-[4-(2-methoxy-ethyl)-piperazin-1-yl]-phenyl}-2,2-dimethyl-propionyl, 3-{3-[4-(2-ethoxy-ethyl)-piperazin-1-yl]-phenyl}-2,2-dimethyl-propionyl, 3-(3-pyrrolidin-1-yl-phenyl)-propionyl, 2-[4-(4-methyl-piperazin-1-yl)-phenyl]-isobutyl, 2-(4-methoxy-phenyl)-acetyl, 2-(3-methoxy-phenyl)-acetyl, 2-[4-(4-methyl-piperazin-1-yl)-phenyl]-acetyl, 2-[4-(4-ethyl-piperazin-1-yl)-phenyl]-acetyl, 2-[4-(4-isopropyl-piperazin-1-yl)-phenyl]-acetyl, 2-(4-pyrrolidin-1-yl-phenyl)-acetyl, 2-[4-(2-diethylamino-ethylamino)-phenyl]-isobutyl, 2-(4-pyrrolidin-1-yl-phenyl)-isobutyl.

Particularly preferred compounds are examples as disclosed in WO 03/020278A1, pp. 17-52, e.g. N-[2-Cyano-4-(2,2-dimethyl-propylamino)-pyrimidin-5-ylmethyl]-4-(1-propyl-piperidin-4-yl)-benzamide, N-[2-Cyano-4-(2,2-dimethyl-propylamino)-pyrimidin-5-ylmethyl]-4-(4-methyl-piperazin-1-yl)-benzamide, N-[2-Cyano-4-(2,2-diethyl-propylamino)-pyrimidin-5-ylmethyl]4-[1-(2-methoxy-ethyl)-piperidin-4-yl]-benzamide, N-[2-Cyano-4-(2,2-dimethyl-propyl amino)-pyrimidin-5-ylmethyl]-4-(4-propyl-piperazin-1-yl)-benzamide, N-[2-Cyano-4-(2,2-dimethyl-propylamino)-pyrimidin-5-ylmethyl]-2,2-dimethyl-3-[4-(4-methyl-piperazin-1-yl)-phenyl]-propionamide, N-[2-Cyano-4-(2,2-dimethyl-propylamino)-pyrimidin-5-ylmethyl]-2,2-dimethyl-3-[3-(4-methyl-piperazin-1-yl)-phenyl]-propionamide, N-[2-Cyano-4-(2,2-dimethyl-propylamino)-pyrimidin-5-ylmethyl]-4-(4-ethyl-piperazin-1-yl)-benzamide, N-[2-Cyano-4-(2,2-dimethyl-propylamino)-pyrimidin-5-ylmethyl]-4-(4-isopropyl-piperazin-1-yl)-benzamide, N-[2-Cyano-4-(2,2-dimethyl-propylamino)-pyrimidin-5-ylmethyl-1]4-[4-(2-ethoxy-ethyl)-piperazin-1-yl]-benzamide, N-[2-Cyano-4-(2,2-dimethyl-propylamino)-pyrimidin-5-ylmethyl]-4-[4-(2-methoxy-ethyl)-piperazin-1-yl]-benzamide, N-[2-Cyano-4-(2,2-dimethyl-propylamino)-pyrimidin-5-ylmethyl]-4-piperazin-1-yl-benzamide, 4-(4-{[2-Cyano-4-(2,2-dimethyl-propylamino)-pyrimidin-5-ylmethyl]-carbamoyl}-phenyl)-piperazine-1-carboxylic acid tert-butyl ester, 4-(3-{[2-Cyano-4-(2,2-dimethyl-propylamino)-pyrimidin-5-ylmethyl]-carbamoyl}-phenyl)-piperazine-1-carboxylic acid tert-butyl ester, N-[2-Cyano-4-(2,2-dimethyl-propylamino)-pyrimidin-5-ylmethyl]-3-(4-methyl-piperazin-1-yl)-benzamide, N-[2-Cyano-4-(2,2-dimethyl-propylamino)-pyrimidin-5-ylmethyl]-3-(4-ethyl-piperazin-1-yl)-benzamide, N-[2-Cyano-4-(2,2-dimethyl-propylamino)-pyrimidin-5-ylmethyl]-3-(4-isopropyl-piperazin-1-yl)-benzamide, N-[2-Cyano-4-(2,2-dimethyl-propylamino)-pyrimidin-5-ylmethyl]-3-[4-(2-methoxy-ethyl)-piperazin-1-yl]-benzamide, N-[2-Cyano-4-(2,2-ethyl-propylamino)-pyrimidin-5-ylmethyl]-3-[4-(2-ethoxy-ethyl)-piperazin-1-yl]benzamide, N-[2-Cyano-4-(2,2-dimethyl-propylamino)-pyrimidin-5-ylmethyl]-4-methoxy-3-(2-pyrrolidin-1-yl-ethoxy)-benzamide, N-[2-Cyano-4-(2,2-dimethyl-propylamino)-pyrimidin-5-ylmethyl]-3-(2-dimethylamino-ethoxy)-4-methoxy-benzamide, N-[2-Cyano-4-(2,2-dimethyl-propylamino)-pyrimidin-5-ylmethyl]-4 dimethylaminomethyl-benzamide, N-[2-Cyano-4-(2,2-dimethyl-propylamino)-pyrimidin-5-ylmethyl]-4-methyl-piperazin-1-ylmethyl)-benzamide, N-[2-Cyano-4-(2,2-dimethyl-propylamino)-pyrimidin-5-ylmethyl]-4-[1-(2-methoxy-ethyl)-piperidin-4-ylmethyl]-benzamide, N-[2-Cyano-4-(2,2-dimethyl-propylamino)pyrimidin-5-ylmethyl]-4-methoxy-3-(2-piperidin-1-yl-ethoxy)-benzamide, N-[2-Cyano-4-(2,2-dimethyl-propylamino)-pyrimidin-5-ylmethyl]-3-[4-(4-ethyl-piperazin-1-yl)-phenyl]-2,2-dimethyl-propionamide or pharmaceutically acceptable salts thereof.

All the cat K inhibitors mentioned above as an alternative class of cat K compounds are known from the literature. This includes their production (see e.g. WO 03/020278A1, pp. 9-12).

Pharmacologically acceptable salts of bisphosphonates and cat K inhibitors are preferably salts with bases, conveniently metal salts derived from groups Ia, Ib, IIa and IIb of the Periodic Table of the Elements, including alkali metal salts, e.g. potassium and especially sodium salts, or alkaline earth metal salts, preferably calcium or magnesium salts, and also ammonium salts with ammonia or organic amines.

Especially preferred pharmaceutically acceptable salts of the cathepsin K inhibitors are maleate salts, e.g. N-[1-(Cyanomethyl-carbamoyl)-cyclohexyl]-4-(1-propyl-piperidin-4-yl)-benzamide hydrogen maleate.

Especially preferred pharmaceutically acceptable salts of the N-bisphosphonates are those where one, two, three or four, in particular one or two, of the acidic hydrogens of the bisphosphonic acid are replaced by a pharmaceutically acceptable cation, in particular sodium, potassium or ammonium, in first instance sodium.

A suitable combination of the present invention is a cathepsin K inhibitor with a N-bisphosphonate. A particularly preferred combination is a N-bisphosphonate as described in this application with a cathepsin K inhibitor as described in this application. More preferred is the combination of any cathepsin K inhibitor with Zoledronic Acid or its pharmaceutically acceptable salt thereof, in particular with Zoledronic Acid. Even more preferred is the combination of a cathepsin K inhibitor as described in this application, e.g. N-[1-Cyanomethyl-carbamoyl)-cyclohexyl]4-(1-propyl-piperidin-4-yl)-benzamide hydrogen maleate.

Administering both therapeutic agents (i.e. cathepsin K inhibitor and N-bisphosphonate) produces an effect that is greater than that of the N-bisphosphonate administered alone. This is advantageous in that it allows for a smaller amount of the N-bisphosphonate to be administered to provide a therapeutic effect. A further advantage is that therapy can be effected for patients who, for example, do not respond adequately to the use of the N-bisphosphonate at what would be considered a maximal strength dose.

According to one aspect of the present invention, there is provided a product comprising a first pharmaceutically acceptable composition containing a N-bisphosphonate and a second pharmaceutically acceptable composition containing a cathepsin K inhibitor for use as a combined preparation for simultaneous, separate or sequential use in treating malignant diseases, e.g. inhibition of bone metastasis, cancer cell growth, induction of cancer cell apoptosis and/or inhibition of tumor-induced bone loss in mammals, e.g. humans.

In one embodiment, the N-bisphosphonate in the first composition is a specific N-bisphosphonate as described in this application. Preferably the N-bisphosphonate in the first composition is Zoledronic Acid or a pharmaceutically acceptable salt thereof. The cathepsin K inhibitors in the second composition may be a cathepsin K inhibitor as defined above. Preferably the cathepsin K inhibitor in the second composition is selected from the examples as disclosed in WO 03/020278A1 (pages 17-52), e.g. N-[2-Cyano-4-(2,2-dimethyl-propylamino)-pyrimidin-5-ylmethyl]-4-(1-propyl-piperidinyl)-benzamide, N-[2-Cyano-4-(2,2-dimethyl-propylamino)-pyrimidin-5-ylmethyl]-4-(4-methyl-piperazin-1-yl)-benzamide, N-[2-Cyano-4-(2,2-dimethyl-propylamino)-pyrimidin-5-ylmethyl][1-(2-methoxy-ethyl)-piperidin-4-yl]-benzamide, N-[2-Cyano-4-(2,2-dimethyl-propylamino)-pyrimidin-5-ylmethyl]-4-(4-propyl-piperazin-1-yl)-benzamide, N-[2-Cyano-4-(2,2-dimethyl-propylamino)-pyrimidin-5-ylmethyl]-2,2-diethyl-3-[4 methyl-piperazin-1-yl)-phenyl]-propionamide, N-[2-Cyano-4-(2,2-dimethyl-propylamino)-pyrimidin-5-ylmethyl]-2,2-dimethyl-3-[3-(4-methyl-piperazin-1-yl)-phenyl]-propionamide, N-[2-Cyano-4-(2,2-dimethyl-propylamino)-pyrimidin-5-ylmethyl]-4-(4-ethyl-piperazin-1-yl)-benzamide, N-[2-Cyano-4-(2,2-dimethyl-propylamino)-pyrimidin-5-ylmethyl]-4-(4-isopropyl-piperazin-1-yl)-benzamide, N-[2-Cyano 2,2-ethyl-propylamino)-pyrimidin-5-ylmethyl]-4-[4-(2-ethoxy-ethyl)-piperazin-1-yl]-benzamide, N-[2-Cyano-4-(2,2-diethyl-propylamino)-pyrimidin-5-ylmethyl])[4-(2-methoxy-ethyl)-piperazin-1-yl]-benzamide, N-[2-Cyano-4-(2,2-dimethyl-propylamino)-pyrimidin-5-ylmethyl]-4-piperazin-1-yl-benzamide, 4-(4-{[2-Cyano 4-(2,2-dimethyl-propylamino)-pyrimidin-5-ylmethyl]-carbamoyl}-phenyl)-piperazine-1-carboxylic acid tert-butyl ester, 4-(3-{[2-Cyano-4-(2,2-dimethyl-propylamino)-pyrimidin-5-ylmethyl]-carbamoyl}-phenyl)-piperazine-1-carboxylic acid tert-butyl ester, N-[2-Cyano-4-(2,2-ethyl-propylamino)-pyrimidin-5-ylmethyl]-3-(4-methyl-piperazin-1-yl)-benzamide, N-[2-Cyano-4-(2,2-dimethyl-propylamino)-pyrimidin-5-ylmethyl]-3-(4-ethyl-piperazin-1-yl)-benzamide, N-[2-Cyano-4-(2,2-diethyl-propylamino)-pyrimidin-5-ylmethyl]-3-(4-isopropyl-piperazin-1-yl)-benzamide, N-[2-Cyano-4-(2,2-dimethyl-propylamino)-pyrimidin-5-ylmethyl]-3-[4-(2-methoxy-ethyl)-piperazin-1-yl]-benzamide, N-[2-Cyano-4-(2,2-dimethyl-propylamino)-pyrimidin-5-ylmethyl]-3-[4-(2-ethoxy-ethyl)-piperazin-1-yl]-benzamide, N-[2-Cyano-4-(2,2-dimethyl-propylamino)-pyrimidin-5-ylmethyl]-4-methoxy-3-(2-pyrrolidin-1-yl-ethoxy)-benzamide, N-[2-Cyano-4-(2,2-dimethyl-propylamino)-pyrimidin-5-ylmethyl]-3-(2-dimethylamino-ethoxy)-4-methoxy-benzamide, N-[2-Cyano-4-(2,2-dimethyl-propylamino)-pyrimidin-5-ylmethyl]-4-dimethylaminomethyl-benzamide, N-[2-Cyano-4-(2,2-dimethyl-propylamino)-pyrimidin-5-ylmethyl]-4-(4-methyl-piperazin-1-ylmethyl)-benzamide, N-[2-Cyano-4-(2,2-dimethyl-propylamino)-pyrimidin-5-ylmethyl]-4-[1-(2-methoxy-ethyl)-piperidin-4-ylmethyl]-benzamide, N-[2-Cyano-4-(2,2-dimethyl-propylamino)-pyrimidin-5-ylmethyl]-4-methoxy-3-(2-piperidin-1-yl-ethoxy)-benzamide, N-[2-Cyano-4-(2,2-diethyl-propylamino)-pyrimidin-5-ylmethyl]-3-[4-(4-ethyl-piperazin-1-yl)-phenyl]-2,2-dimethyl-propionamide or pharmaceutically acceptable salts thereof; or is N-[1-(Cyanomethyl-carbamoyl)-cyclohexyl]-4-(1-propyl-piperidin-4-yl)-benzamide or a pharmaceutically acceptable salt thereof, e.g. the hydrogen maleate salt.

The present invention provides for the administering of each of the antagonists separately but as part of the same therapeutic treatment program or regimen, and it is contemplated that separate administration of each compound, at different times and by different routes, will sometimes be recommended. Thus the two components need not necessarily be administered at essentially the same time. In another embodiment the N-bisphosphonate, e.g. the Zoledronic Acid, will be given several days prior to initiation of the cathepsin K inhibitor either daily, monthly, 3-monthly or yearly or “on demand”. If co-administered separately, it is also preferred that the N-bisphosphonate component be administered in an injectable and the cathepsin K inhibitor in an oral dosage form.

The product may comprise a kit. The kit may comprise a container for containing the separate compositions such as a divided bottle or a divided foil packet, wherein each compartment contains a plurality of dosage forms (e.g., tablets) comprising either the alpha1-adrenoceptor antagonist or the muscarinic antagonist.

Alternatively, rather than separating the active ingredient-containing dosage forms, the kit may contain separate compartments each of which contains a whole dosage which comprises separate compositions. An example of this type of kit is a blister pack wherein each individual blister contains the dosage units of the two drugs, e.g. one bottle comprises lyophilized N-bisphosphonate and one table comprising the cathepsin K inhbitor.

Typically the kit comprises directions for the administration of the separate components. Such instructions would cover situations such as:

i) the dosage form in which the components are administered (e.g. oral and parenteral),

ii) when the component parts of the product are administered at different dosage intervals, or

iii) when titration of the individual components of the combination is desired by the prescribing physician.

An example of such a kit is a so-called blister pack. Blister packs are well known in the packaging industry and are widely used for the packaging of pharmaceutical unit dosage forms such as tablets, capsules, bottles and the like. Blister packs generally consist of a sheet of relatively stiff material covered with a foil of a preferably transparent plastic material. During the packaging process recesses are formed in the plastic foil. The recesses have the size and shape of the tablets or capsules to be packed. Next, the tablets or bottles are placed in the recesses and the sheet of relatively stiff material is sealed against the plastic foil at the face of the foil which is opposite from the direction in which the recesses between the plastic foil and the sheet. Preferably, the strength of the sheet is such that the tablets or bottles can be removed from the blister pack by manually applying pressure on the recesses whereby an opening is formed in the sheet at the place of the recess. Tablet(s) or bottle(s) can then be removed via said opening.

It may be desirable to provide a memory aid on the kit, e.g., in the form of numbers next to the tablets or capsules whereby the numbers correspond with the days of the regimen during which the tablets or capsules so specified should be ingested. Another example of such a memory aid is a calendar printed on the car, e.g. as follows “First Week, Monday, Tuesday, . . . etc. . . . Second Week, Monday, Tuesday, . . . ”, etc. Other variations of memory aids will be readily apparent. A “daily dose” can be a single tablet or capsule or several pills or capsules to be taken on a given day. Also a daily dose of the first compound can consist of one tablet or capsule while a daily dose of the second compound can consist of several tablets or capsules and vice versa. The memory aid should reflect this.

The N-bisphosphonate pharmaceutical compositions may be, for example, compositions for enteral, such as oral, rectal, aerosol inhalation or nasal administration, compositions for parenteral, such as intravenous or subcutaneous administration, or compositions for transdermal administration (e.g. passive or iontophoretic). Preferably, the N-bisphosphonate pharmaceutical compositions are adapted to parenteral (especially intravenous, intra-arterial or transdermal) administration. Intravenous administration is considered to be of particular importance. Preferably the N-bisphosphonate active ingredient is in a parenteral form, most preferably an intravenous form. Preferably, the cathepsin K inhibitor pharmaceutical compositions are adapted to oral administration.

Normally the dosage is such that a single dose of the bisphosphonate active ingredient from 0.002-20.0 mg/kg, especially 0.01-10.0 mg/kg, is administered to a warm-blooded animal weighing approximately 75 kg, e.g. the preferred doses for Zoledronic Acid are either a 4 mg or 5 mg once per month per adult human (but may alter dependent on the kind and state of the disease). If desired, this dose may also be taken in several, optionally equal, partial doses.

“mg/kg” means mg drug per kg body weight of the mammal—including man—to be treated.

The dose mentioned above—either administered as a single dose (which is preferred) or in several partial doses—may be repeated, for example once daily, once weekly, once every month, once every three months, or once every year. In other words, the pharmaceutical compositions may be administered in regimens ranging from continuous daily therapy to intermittent cyclical therapy.

Preferably, the N-bisphosphonates are administered in doses which are in the same order of magnitude as those used in the treatment of the malignant diseases classically treated with bisphosphonic acid derivatives, such as tumour-induced hypercalcemia or bone metastases of MM or breast cancer. In other words, preferably the N-bisphosphonic acid derivatives are administered in doses which would likewise be therapeutically effective in the treatment of tumour-induced hypercalcaemia or bone metastases or breast cancer, i.e. preferably they are administered in doses which would likewise effectively inhibit bone resorption and metastases invasion and growth.

Formulations in single dose unit form contain preferably from about 1% to about 90%, and formulations not in single dose unit form contain preferably from about 0.1% to about 20%, of the active ingredient. Single dose unit forms for oral administration such as capsules, tablets or dragées contain e.g. from about 1 mg to about 500 mg of the active ingredient.

Pharmaceutical preparations for enteral and parenteral administration are, for example, those in dosage unit forms, such as dragées, tablets or capsules and also ampoules. They are prepared in a manner known per se, for example by means of conventional mixing, granulating, confectioning, dissolving or lyophilising processes.

For example, pharmaceutical preparations for oral administration can be obtained by combining the active ingredient with solid carriers, where appropriate granulating a resulting mixture, and processing the mixture or granulate, if desired or necessary after the addition of suitable adjuncts, into tablets or dragée cores. Suitable carriers are especially fillers, such as sugars, for example lactose, saccharose, mannitol or sorbitol, cellulose preparations and/or calcium phosphates, for example tricalcium phosphate or calcium hydrogen phosphate, and also binders, such as starch pastes, using, for example, corn, wheat, rice or potato starch, gelatin, tragacanth, methylcellulose and/or polyvinylpyrrolidone and, if desired, disintegrators, such as the above-mentioned starches, also carboxymethyl starch, crosslinked polyvinylpyrrolidone, agar or alginic acid or a salt thereof, such as sodium alginate. Adjuncts are especially flow-regulating agents and lubricants, for example silicic acid, talc, stearic acid or salts thereof, such as magnesium or calcium stearate, and/or polyethylene glycol. Dragee cores are provided with suitable coatings that may be resistant to gastric juices, there being used, inter alia, concentrated sugar solutions that optionally contain gum arabic, talc, polyvinylpyrrolidone, polyethylene glycol and/or titanium dioxide, or lacquer solutions in suitable organic solvents or solvent mixtures or, to produce coatings that are resistant to gastric juices, solutions of suitable cellulose preparations, such as acetylcellulose phthalate or hydroxypropylmethyl-cellulose phthalate. Colouring substances or pigments may be added to the tablets or dragee coatings, for example for the purpose of identification or to indicate different doses of active ingredient. Other orally administrable pharmaceutical preparations are dry-filled capsules made of gelatin, and also soft, sealed capsules made of gelatin and a plasticiser, such as glycerol or sorbitol. The dry-filled capsules may contain the active ingredient in the form of a granulate, for example in admixture with fillers, such as lactose, binders, such as starches, and/or glidants, such as talc or magnesium stearate, and, where appropriate, stabilisers. In soft capsules the active ingredient is preferably dissolved or suspended in suitable liquids, such as fatty oils, paraffin oil or liquid polyethylene glycols, it being possible also for stabilisers to be added.

Parenteral formulations are especially injectable fluids that are effective in various manners, such as intravenously, intra-arterially, intramuscularly, intraperitoneally, intranasally, intradermally, subcutaneously, preferably intravenously. Such fluids are preferably isotonic aqueous solutions or suspensions which can be prepared before use, for example from lyophilised preparations which contain the active ingredient alone or together with a pharmaceutically acceptable carrier. The pharmaceutical preparations may be sterilised and/or contain adjuncts, for example preservatives, stabilisers, wetting agents and/or emulsifiers, solubilisers, salts for regulating the osmotic pressure and/or buffers. Preferred parenteral forms are intravenous infusion solutions, preferably containg from about 1 mg up to about 20 mg of active substance per unit dose; for instance in an infusion solution volume of from about 5 up to about 200 ml, e.g. for infusion over a period of from about 1 minute up to about 1 hour or more. Such preferred parenteral forms are typically administered at intervals of from about once per week up to one year.

The cat K pharmaceutical compositions of the invention may be, for example, compositions for enteral, such as oral, rectal, aerosol inhalation or nasal administration, compositions for parenteral, such as intravenous or subcutaneous administration, or compositions for transdermal administration (e.g. passive or iontophoretic).

Preferably, the cat K pharmaceutical compositions of the invention are adapted to oral or parenteral (especially oral) administration. Intravenous and oral, first and foremost oral, adminstration is considered to be of particular importance.

The particular mode of administration and the dosage may be selected by the attending physician taking into account the particulars of the patient, especially age, weight, life style, activity level, and disease state as appropriate. Preferably, however, the cat K pharmaceutical compositions are administered orally in a twice or once daily dosage regimen.

The dosage of cat K inhibitor of the invention administered is dependent on the species of warm-blooded animal (mammal), the body weight, age and individual condition, and on the form of administration. A unit dosage for oral administration to a mammal of about 50 to 70 kg may contain between about 0.05 and 5000 mg, e.g. from 0.5-500 mg, of the active ingredient. Preferably the dosage for once daily oral administering N-[1-(Cyanomethyl-carbamoyl)-cyclohexyl]-4-(1-propyl-piperidin-4-yl)-benzamide is between 5 and 50 mg.

Cat K inhibitor formulations of the invention in single dose unit form contain preferably from about 1% to about 90%, and formulations not in single dose unit form contain preferably from about 0.1% to about 20%, of the active ingredient. Single dose unit forms such as capsules, tablets or dragées contain e.g. from about 0.05 mg to about 5000 mg of the active ingredient.

Cat K inhibitor pharmaceutical preparations of the invention for enteral and parenteral administration are, for example, those in dosage unit forms, such as dragées, tablets or capsules and also ampoules. They are prepared in a manner known per se, for example by means of conventional mixing, granulating, confectioning, dissolving or lyophilising processes.

For example, pharmaceutical preparations for oral administration can be obtained by combining the active ingredient with solid carriers, where appropriate granulating a resulting mixture, and processing the mixture or granulate, if desired or necessary after the addition of suitable adjuncts, into tablets or dragée cores. Other orally administrable pharmaceutical preparations are dry-filled capsules made of gelatin, and also soft, sealed capsules made of gelatin and a plasticiser, such as glycerol or sorbitol. The dry-filled capsules may contain the active ingredient in the form of a granulate, for example in admixture with fillers, such as lactose, binders, such as starches, and/or glidants, such as talc or magnesium stearate, and, where appropriate, stabilisers. In soft capsules the active ingredient is preferably dissolved or suspended in suitable liquids, such as fatty oils, paraffin oil or liquid poly-ethylene glycols, it being possible also for stabilisers to be added.

Parenteral formulations are especially injectable fluids that are effective in various manners, such as intravenously, intramuscularly, intraperitoneally, intranasally, intradermally or subcutaneously. Such fluids are preferably isotonic aqueous solutions or suspensions which can be prepared before use, for example from lyophilised preparations which contain the active ingredient alone or together with a pharmaceutically acceptable carrier. The pharmaceutical preparations may be sterilised and/or contain adjuncts, for example preservatives, stabilisers, wetting agents and/or emulsifiers, solubilisers, salts for regulating the osmotic pressure and/or buffers.

The following examples are intended to illustrate the invention and are not to be construed as being limitations thereon.

### EXAMPLES

### Example 1

### Formulations

Preparation of formulations: N-[1-(Cyanomethyl-carbamoyl)-cyclohexyl]-4-[4-(1-propyl)-piperazin-1-yl]-benzamide or its hydrogen maleate salt is dissolved in a mixture of 1-Methyl-2pyrrilidone (NMP) and Polyethylene glycol 300 (PEG300) (1:10, vol:vol) (both Fluka Chemica, Buchs, Switzerland). Zoledronic Acid is dissolved in sterile 0.9% aqueous saline solution (B. Braun Medical AG, Emmenbrlcke, Switzerland).

### Example 2

### Osteolytic activity of combinations of a specific cat K inhibitory compound and a specific bisphosphonate compound in the 4T1_{Luc2000} Assay

Method

Intra-Tibial Injection of 4T1_{Luc2000} Mouse Mammary Carcinoma Cells:

Athymic Balb\c nude, female mice (Spezialzuchten, Stein, Switzerland) are anaesthetized by i.p. administration of 1011/kg ketarom: a mixture of 100 mg/kg Ketalar® 50 (Parker-Davis, Zurich, Switzerland) and 10 mg/kg xylazine (Rompun®, Bayer, Lyssach, Switzerland). Using a 30 gauze needle a cell suspension of 1.25×105 in 201 of HBSS (Invitrogen, Basel Switzerland) is injected through the articular cartilage and epiphysis into the tibia. In total, 48 animals in 6 groups, 8 animals each, are used in this study. Treatment compounds and doses are: Zoledronic acid 100 μg/kg, s.c. twice weekly and N-[1-(Cyanomethyl-carbamoyl)-cyclohexyl]-4-(1-propyl-piperidin-4-yl)-benzamide or its hydrogen maleate salt 50 mg/kg, p.o., twice daily for 7 days. Group1 is control 1: tumor cells/vehicle treated. Group2: Zoledronic Acid. Group3: N-[1-(Cyanomethyl-carbamoyl)-cyclohexyl]-4-(1-propyl-piperidin-4-yl)-benzamide or its hydrogen maleate salt. Group4: combination of Zoledronic Acid and N-[1-Cyanomethyl-carbamoyl)-cyclohexyl]-4-(1-propyl-piperidin-4-yl)-benzamide or its hydrogen maleate salt. Group5 is control 3: heat inactivated tumour cells, no compound treatment. Group6 is control 2: no tumor cells.

IVIS Xenogen Imaging, Twice Weekly:

Firefly D-Luciferin (1×10⁵ cells) potassium salt solution (enogen Biosciences, Cranbury, N.J., USA) is injected into the animals (100 mg/kg, i.v. in PBS) 5 min prior to anaesthetization by i.p. administration of 10 ml/kg ketarom. Imaging is performed 5 min following Ketarom administration. Imaging parameters are: 1 min exposure with binning 8 by filter/stop/filter open. The LivingImage software is used to analyze the images obtained.

Peripheral Quantitative Computed Tomography (pQCT), End-Point Determination:

Total, cortical, and cancellous bone mass and geometry are monitored using an XCT-Research SA+ (Stratec-Norland, Pforzheim, Germany) fitted with a 0.5 mm collimator. The following set-up is chosen for the measurements: voxel-size: 0.1 mm×0.1 mm×0.5 mm, scan speed: scout view 10 mm/s, final scan 2 mm/s, 1 block, contour mode 1, peel mode 2, cortical threshold 350 mg/cm3, inner threshold 350 mg/cm3. Slices located 2, 3, 4, and 5 mm distal from the intercondylar tubercle in the proximal tibia metaphysis are analysed.

MicroCT Measurements with VivaCT40 5 Days Before, 1 Week After and 2 Weeks Post Treatment Start:

3D structural parameters of cancellous bone are measured non-invasively by microCT as described. Mice are anaesthetized with Forene and their hind-limb firmly is fixed on a mouse tray for measurement in the vivaCT40 (SCANCO Medical, Bassersdorf, Switzerland). A region of 200 slices at a position of 1 mm below the growth plate (secondary spongiosa) corresponding to the site of injection of the 4T1luc2000 cells is measured at a nominal resolution of approximately 15 μm (FIG. 3). Measurements will provide direct information on the osteolytic activity of the tumor cells on structural parameters like the cancellous bone volume, number, thickness, separation as well as provide information on connectivity (connectivity density, structure model index). In addition images showing progression of tumor growth over time and the therapeutic effects of the compounds are available.

Dual-Energy X-Ray Absorptiometry (DEXA), End-Point Determination:

Tibial bone mineral content (BMC, mg) and bone mineral density (BMD, mg/cm²) are measured ex vivo at necropsy using a Hologic QDR-1000 instrument (Hologic, Waltham, Mass., USA) adapted for measurement of small animals. Ultrahigh resolution mode (line spacing 0.0254 cm, resolution 0.0127 cm) and a 0.9 cm diameter collimator are used. The excised long-bones are placed in 70% alcohol onto a resin platform provided by the Hologic for soft tissue calibration. The total (L1-L3) and the proximal third (L1) of the injected tibia are measured.

Statistical Analysis:

All results from the bone studies are expressed as mean *standard error (SEM). Data will be subjected to one-way analysis of variance (ANOVA). The Levene F-test is used to test equality of variances, and differences between groups tested by Dunnett test (significance level: * p<0.05). All statistical tests are two-tailed. Compound-treated, tumor bearing groups are tested for differences from vehicle treated, tumor-bearing animals.

If administered in combination with Zoledronic Acid (100 ug/kg s.c. twice weekly), N-[1-(Cyanomethyl-carbamoyl)-cyclohexyl]-4-(1-propyl-piperidin-4-yl)-benzamide or its hydrogen maleate salt (50 mg/kg p.o. twice daily) is expected to exhibit an additive anti-osteolytic activity which is superior to Zoledronic Acid single treatment.

### Example 3

### Combinations of a Specific Cat K Inhibitory Compound and a Specific Bisphosphonate Compound in the PC-3M2AC6 Prostate Experimental Bone Metastasis Model

Luciferase-Expressing Human Prostate Cancer Cell Line, PC-3M2AC6:

Metastatic human prostate cancer cell line, PC-3M (obtained from Prof. J. I. Fidler, M. D. Anderson Cancer Center, Houston, Tex.), is transfected with a firefly luciferase expression vector, pGL-3 (Promega) in the laboratories of Xenogen Corporation to produce a clone, designated PC-3M2AC6, which is selected based on high light output in the presence of luciferin, and retention of in vitro sensitivity to the antiproliferative activity of cytotoxic drugs. The PC-3M2AC6 cell line is maintained and expanded for implantation in RPMI 1640 medium containing 10% heat-inactivated FBS (both from Life Technologies, Grand Island, N.Y.). For injection, cells are harvested at 90% confluency by a brief trypsinization with 0.25% trypsin containing 1 mM EDTA (Life Technologies, Grand Island, N.Y.). After the cell suspension is collected, trypsin is immediately inactivated with HBSS containing 10% FBS. Cells are washed once with HBSS, and are suspended in HBSS at 30 million cells/mL for implantation. The viability of cells in single-cell suspension used for injection is >90% (by trypan blue exclusion).

Animals

Male athymic (nu/nu) nude mice are purchased from the Charles River Laboratories, Wilmington, Mass. The mice are identified via ear markings and housed 4/cage under pathogen-free conditions and are used at 6-8 weeks of age. Twelve mice are used per treatment group in each experiment.

Intracardiac Injection of PC-3M2AC6 Cells

Intracardiac injection of PC-3M2AC6 cells results in colonization of bones. Injection of these cells into the left cardiac ventricle requires abdominal surgery in order to expose the heart through the diaphragm. Prior to surgery mice are anesthetized with a single intraperitoneal injection of a freshly prepared mixture of ketamine hydrochloride (Ketaseti, 150 mg/kg) and xylazine (Rompun™, 12 mg/kg). A 10 mm upper midline vertical incision is created, and the liver is retracted to visualize the base of the heart through the diaphragm. Tumor cell suspensions (3×10⁶ cells in 100 μL HBSS in the first two experiments, and 2×10⁶ cells in 100 μL HBSS in the third experiment) are injected into the left ventricle through the diaphragm using a 27G½ or 28G½ needle. After the injection the abdominal incision is closed with 3-5 metal wound clips. Each animal receives a single dose of 0.1 mg/kg butorphenol (Torbugesic™), and is transferred onto a heating pad (37° C.-42° C.) to recover from the anaesthesia. After recovery all animals are transferred to their cages. A total number of 150 mice per experiment are injected with tumor cells. A small percentage of animals (<5%) may die within one week after the surgery due to post-surgical complications. Ten days (7 days in the third experiment) after tumor cell injection all surviving animals are imaged as described below and mice with suitable tumors are selected for dosing and sorted into groups of 12. The sorting process produces groups balanced with respect to mean and range of tumor burden.

Formulations and Dosing

The compound (a cat K inhibitor of formula VII, e.g. one of the following compounds: N-(2-Cyano-4-(2,2-dimethyl-propylamino)-pyrimidin-5-ylmethyl]-4-(1-propyl-piperidin-4-yl)-benzamide, N-[2-Cyano-4-(2,2-dimethyl-propylamino)-pyrimidin-5-ylmethyl]-4-(4-methyl-piperazin-1-yl)-benzamide, N-[2-Cyano-4-(2,2-dimethyl-propylamino)-pyrimidin-5-ylmethyl])[1-(2-methoxy-ethyl)-piperidin-4-yl]-benzamide, N-[2-Cyano-4-(2,2-dimethyl-propylamino)-pyrimidin-5-ylmethyl]-4-(4-propyl-piperazin-1-yl)-benzamide, N-[2-Cyano-4-(2,2-dimethyl-propylamino)-pyrimidin-5-ylmethyl]-2,2-dimethyl-3-[4-(4-methyl-piperazin-1-yl)-phenyl]-propionamide, N-[2-Cyano-4-(2,2-dimethyl-propylamino)-pyrimidin-5-ylmethyl]-2,2-ethyl-3-[3-(4-methyl-piperazin-1-yl)-phenyl]-propionamide, N-[2-Cyano-4-(2,2-dimethyl-propyl amino)-pyrimidin-5-ylmethyl]-4-(4-ethyl-piperazin-1-yl)-benzamide, N-[2-Cyano-4-(2,2-dimethyl-propylamino)-pyrimidin-5-ylmethyl]-4-(4-isopropyl-piperazin-1-yl)-benzamide, N-[2-Cyano-4-(2,2-dimethyl-propylamino)-pyrimidin-5-ylmethyl]-4-[4-(2-ethoxy-ethyl)-piperazin-1-yl]-benzamide, N-[2-Cyano-4-(2,2-dimethyl-propylamino)-pyrimidin-5-ylmethyl]-4-[4-(2-methoxy-ethyl)-piperazin-1-yl]-benzamide, N-[2-Cyano-4-(2,2-dimethyl-propylamino)-pyrimidin-5-ylmethyl]-4-piperazin-1-yl-benzamide, 4-(4-{[2-Cyano-4-(2,2-dimethyl-propylamino)-pyrimidin-5-ylmethyl]-carbamoyl}-phenyl)-piperazine-1-carboxylic acid tert-butyl ester, 4-(3-{[2-Cyano-4-(2,2-diethyl-propylamino)-pyrimidin-5-ylmethyl]-carbamoyl}-phenyl)-piperazine-1-carboxylic acid tert-butyl ester, N-[2-Cyano-4-(2,2-dimethyl-propylamino)-pyrimidin-5-ylmethyl]-3-(4-methyl-piperazin-1-yl)-benzamide, N-[2-Cyano-4-(2,2-dimethyl-propylamino)-pyrimidin-5-ylmethyl]-3-(4-ethyl-piperazin-1-yl)-benzamide, N-[2-Cyano-4-(2,2-dimethyl-propylamino)-pyrimidin-5-ylmethyl]-3-(4-isopropyl-piperazin-1-yl)-benzamide, N-[2-Cyano-4-(2,2-dimethyl-propylamino)-pyrimidin-5-ylmethyl]-3-[4-(2-methoxy-ethyl)-piperazin-1-yl]-benzamide, N-[2-Cyano-4-(2,2-dimethyl-propylamino)-pyrimidin-5-ylmethyl]-3-[4-(2-ethoxy-ethyl)-piperazin-1-yl]-benzamide, N-[2-Cyano-4-(2,2-dimethyl-propylamino)-pyrimidin-5-ylmethyl]-4-methoxy-3-(2-pyrrolidin-1-yl-ethoxy)-benzamide, N-[2-Cyano-4-(2,2-diethyl-propylamino)-pyrimidin-5-ylmethyl]-3-(2-dimethylamino-ethoxy)-4-methoxy-benzamide, N-[2-Cyano-4-(2,2-dimethyl-propylamino)-pyrimidin-5-ylmethyl]-4-dimethylaminomethyl-benzamide, N-[2-Cyano-4-(2,2-dimethyl-propylamino)-pyrimidin-5-ylmethyl](4-methyl-piperazin-1-ylmethyl)-benzamide, N-[2-Cyano-4-(2,2-dimethyl-propylamino)-pyrimidin-5-ylmethyl]-4-[1-(2-methoxy-ethyl)-piperidin-4-ylmethyl]-benzamide, N-[2-Cyano-4-(2,2-dimethyl-propylamino)-pyrimidin-5-ylmethyl]-4-methoxy-3-(2-piperidin-1-yl-ethoxy)-benzamide, N-[2-Cyano-4-(2,2-dimethyl-propylamino)-pyrimidin-5-ylmethyl]-3-[4-(4-ethyl-piperazin-1-yl)-phenyl]-2,2-dimethyl-propionamide) is formulated as a suspension in 1% carboxymethyl cellulose, and is dosed orally, once daily, for 5 days/week at 100 mg/kg. Zoledronic Acid may be dissolved in sterile 0.9% aqueous saline solution (B. Braun Medical AG, Emmenbrücke, Switzerland) and may be dosed parenterally 100 ug/kg s.c. twice weekly.

Non-Invasive Detection and Quantitation of Bone Metastasis

Luciferase-expressing PC-3M2AC6 tumors are visualized using a non-invasive IVIS imaging system. LivingImage™ v.2.11 software is used to quantify the light output. Mice are imaged ingroups of 4. First, animals are injected intravenously with 50 mg/kg of D-luciferin (potassium salt) and then are anesthetized with a single intraperitoneal injection of a freshly prepared mixture of ketamine hydrochloride (Ketaset™, 150 mg/kg) and xylazine (Rompun™, 12 mg/kg). Anesthetized mice are placed in the supine position in the imaging chamber, and a regular photograph of the animals is taken first. Recording of the light emission always starts 15 minutes after the injection of D-luciferin. All images are acquired for 1 minute. The images are then superimposed on the regular photographs of the mice to give composite pictures. Images are subsequently analyzed for light output using the LivingImage™ v.2.11software.

Calculations of Results

In the first two experiments tumors in the mandible area are quantified for each image. In the third experiment tumors in tibias and femurs (combined) are used for analysis. Antitumor activity (% T/C) is expressed as % ΔT/ΔC (comparing Δ tumor mean photon counts for treatment group to vehicle control group at the end of the experiment).

### Example 4

### Combination of a Specific Cat K Inhibitory Compound and a Specific Bisphosphonate Compound in a Clinical Study

Objectives of the Study:

One of the objectives is to determine the safety profile of increasing doses of N-[1-(Cyanomethyl-carbamoyl)-cyclohexyl]-4-(1-propyl-piperidin-4-yl)-benzamide in combination with Zoledronic Acid 4 mg vs. Zoledronic Acid 4 mg alone and versus N-[1-(Cyanomethyl-carbamoyl)-cyclohexyl]-4-(1-propyl-piperidinyl)-benzamide alone in cancer patients with bone metastases.

Another objective is to assess the efficacy on biochemical parameters of bone resorption and formation in cancer patients with bone metastases. Within the efficacy parameters, urinary NTX (measured as urinary creatinine/NTX ratio) will be the primary efficacy variable and serum NTX and CTX will be secondary efficacy variables. In addition, effects on pain (Brief pain inventory), and performance status (ECOG-score) will be studied.

Overall Study Design

The study is an open-label, randomized, multicenter, fixed dose escalation trial of three increasing dose intensities of o.d. oral N-[1-Cyanomethyl-carbamoyl)-cyclohexyl]-4-(1-propyl-piperidin-4-yl)-benzamide tablets (10 mg, 25 mg, 50 mg) in combination with Zoledronic Acid 4 mg vs. intravenous Zoledronic Acid 4 mg alone vs. N-[1-Cyanomethyl-carbamoyl)-cyclohexyl]4-(1-propyl-piperidin-4-yl)-benzamide in the 25 mg and 50 mg o.d. dose in cancer patients with documented bone metastases. All patients will receive oral calcium and vitamin D supplementation during the trial. Each patient will be followed for 12 weeks. Treatment after that trial period is to the discretion of the investigator (Zoledronic Acid 4 mg (=Zometa® 4 mg) is approved for bone metastases from all solid tumors and bone lesions from multiple myeloma).

Overall, 102 patients will be recruited to randomize 17 patients for each of the following 6 treatment arms (all patients will receive oral calcium and vitamin D supplementation):
1. ZOMETA 4 mg i.v. at days 0, 21, 42, and 63 AND
   N-[1-Cyanomethyl-carbamoyl)-cyclohexyl]-4-(1-propyl-piperidin-4-yl)-benzamide 10 mg p.o., o.d. from day 0 to day 84
2. ZOMETA 4 mg i.v. at days 0, 21, 42, and 63 AND
   N-[1-Cyanomethyl-carbamoyl)-cyclohexyl]-4-(1-propyl-piperidinyl)-benzamide 25 mg p.o., o.d. from day 0 to day 84
3. ZOMETA 4 mg i.v. at days 0, 21, 42, and 63 AND
   N-[1-Cyanomethyl-carbamoyl)-cyclohexyl]-4-(1-propyl-piperidin-4-yl)-benzamide 50 mg p.o., o.d. from day 0 to day 84
4. N-[1-(Cyanomethyl-carbamoyl)-cyclohexyl])-4-(1-propyl-piperidin-4-yl)-benzamide 25 mg p.o., o.d. from day 0 to day 84
5. N-[1-(Cyanomethyl-carbamoyl)-cyclohexyl]-4-(1-propyl-piperidinyl)-benzamide 50 mg p.o., o.d. from day 0 to day 84
6. ZOMETA 4 mg i.v. at days 0, 21, 42, and 63 (control arm)

17 patients will be entered into each dosing group level resulting in 15 evaluable patients per group allowing 2 dropouts per group. Each group will include at least 5 patients with breast cancer, 5 patients with hormone-refractory prostate cancer and 7 patients with any tumor entity.

ZOMETA 4 mg will be given as an intravenous infusion over at least 15 min in a final infusion volume of not less than 100 mL. ZOMETA must not be given together with solutions containing Ca²⁺ or other divalent cations. No restrictions are made regarding the oral intake of N-[1-(Cyanomethyl-carbamoyl)-cyclohexyl]-4-(1-propyl-piperidin-4-yl)-benzamide, except that the time of intake should be the same over the treatment period. Each patient will then be followed for 12 weeks to collect safety data and bone resorption and formation marker data.

Schematic design diagram

| | | Randomization and Evaluation, | |
|---|---|---|---|
| Period | Screening | groups (1) to (6) | Evaluation, groups (1) to (6) |
| Visit | 1 | 2 | 3, 4, 5, 6, 7 |
| Week | −2 weeks | 0 | 1, 3, 6, 9, 12 |
| Treatment* | none | N-[1-(Cyanomethyl-carbamoyl)- | N-[1-(Cyanomethyl-carbamoyl)- |
| | | cyclohexyl]-4-(1-propyl-piperidin-4- | cyclohexyl]-4-(1-propyl- |
| | | yl)-benzamide*** + Zometa** | piperidin-4-yl)-benzamide + Zometa** |
| | | (groups 1-3) | (groups 1-3) |
| | | N-[1-(Cyanomethyl-carbamoyl)- | N-[1-(Cyanomethyl-carbamoyl)- |
| | | cyclohexyl]-4-(1-propyl-piperidin-4- | cyclohexyl]-4-(1-propyl- |
| | | yl)-benzamide alone (groups 4-5) | piperidin-4-yl)-benzamide alone |
| | | Zometa** alone (group 6) | (groups 4-5) |
| | | | Zometa** alone (group 6) |

*All patients will receive oral calcium and vitamin D supplementation (see section 3.4.2, Calcium and vitamin supplements).
**ZOMETA 4 mg is given every 3 weeks (i.e., on visits 2, 4, 5 and 6) as a single infusion (100 ml) over no less than 15 minutes.
***Composition of placebo and N-[1-(Cyanomethyl-carbamoyl)-cyclohexyl]-4-(1-propyl-piperidin-4-yl)-benzamide comprising hard gelatin capsules (mg) is given once a day without restriction to time of administration

| | Placebo | 5 mg | 25 mg | 50 mg |
|---|---|---|---|---|
| N-[1-(Cyanomethyl- | — | ⁽¹⁾6.41 | ⁽²⁾32.05 | ⁽³⁾64.1 |
| carbamoyl)- | | | | |
| cyclohexyl]-4-(1-propyl- | | | | |
| piperidin-4-yl)- | | | | |
| benzamide hydrogen | | | | |
| malete | | | | |
| Lactose | 210.6 | 276.2 | 250.55 | 218.5 |
| Starch | 144.0 | — | — | — |
| Pregelatinized starch | — | 72.0 | 72.0 | 72.0 |
| Colloidal anhydrous silica | 1.8 | 1.8 | 1.8 | 1.8 |
| Magnesium stearate | 3.6 | 3.6 | 3.6 | 3.6 |
| Total weight of capsule fill | 360.0 | 360.0 | 360.0 | 360.0 |

⁽¹⁾corresponding to 5 mg free base
⁽²⁾corresponding to 25 mg free base
⁽³⁾corresponding to 50 mg free base

1. A pharmaceutical preparation which comprises a bisphosphonate of formula I, or a physiologically acceptable and -cleavable ester or a salt thereof 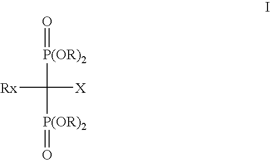
wherein
X is hydrogen, hydroxyl, amino, alkanoyl, or an amino group substituted by C₁-C₄ alkyl, or alkanoyl;
R is hydrogen or C₁-C₄ alkyl and
Rx is a side chain which contains an optionally substituted amino group, or a nitrogen containing heterocycle (including aromatic nitrogen-containing heterocycles),
or a pharmaceutically acceptable salt thereof or any hydrate thereof;
in combination with one of the following:
a) a cat K inhibitor of formula V, or a physiologically acceptable and -cleavable ester or a salt thereof 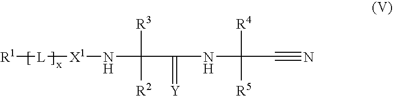
wherein R¹ is optionally substituted (aryl, aryl-lower alkyl, lower alkenyl, lower alkynyl, heterocyclyl or heterocyclyl-lower alkyl);
R² and R³ together represent lower alkylene, optionally interrupted by O, S or NR⁶, so as to form a ring with the carbon atom to which they are attached, and R⁶ is hydrogen, lower alkyl or aryl-lower alkyl;
R⁴ and R⁵ are independently H, or optionally substituted (lower alkyl or aryl-lower alkyl), —C(O)OR⁷, or —C(O)NR⁷R⁸, wherein R⁷ is optionally substituted (lower alkyl, aryl, aryl-lower alkyl, cycloalkyl, bicycloalkyl, bicycloalkyl or heterocyclyl), and R⁸ is H, or optionally substituted (lower alkyl, aryl, aryl-lower alkyl, cycloalkyl, bicycloalkyl, bicycloalkyl or heterocyclyl); or
R⁴ and R⁵ together represent lower alkylene, optionally interrupted by O, S or NR⁶, so as to form a ring with the carbon atom to which they are attached, and R⁶ is hydrogen, lower alkyl or aryl-lower alkyl; or
R⁴ is H or optionally substituted lower alkyl and R⁵ is a substituent of formula —X²—(Y¹)ₙ—(Ar)ₚ-Q-Z wherein
Y¹ is O, S, SO, SO₂, N(R⁶)SO₂, N—R⁶, SO₂NR⁶, CONR⁶ or NR⁶CO;
N is zero or one;
P is zero or one;
X² is lower alkylene: or when n is zero, X² is also C₂-C₇-alkylene interrupted by O, S, SO, SO₂, NR⁶, SO₂NR⁶, CONR⁶ or NR⁶CO, and R⁶ is hydrogen, lower alkyl or aryl-lower alkyl;
Ar is arylene;
Z is hydroxyl, acyloxy, carboxyl, esterified carboxyl, amidated carboxyl, aminosulfonyl, (lower alkyl or aryl-lower alkyl)aminosulfonyl, or (lower alkyl or aryl-lower alkyl)sufonylaminocarbonyl; or Z is tetrazolyl, triazolyl or imidazolyl;
Q is a direct bond, lower alkylene, Y¹-lower alkylene or C₂-C₇-alkylene interrupted by Y¹;
X¹ is —C(O)—, —C(S)—, —S(O)—, —S(O)₂—, or —P(O)(OR⁶)—, and R⁶ is as defined above;
Y is oxygen or sulphur;
L is optionally substituted -Het-, -Het-CH₂— or —CH₂-Het-, and Het is a hetero atom selected from O, N or S; and
X is zero or one; and
aryl in the above definitions represents carbocyclic or heterocyclic aryl; or
b) a cat K inhibitors of formula VII, or a physiologically acceptable and -cleavable ester or a salt thereof 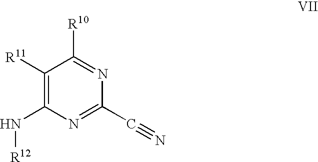
wherein
R¹⁰ is H, —R⁴, —OR¹⁴ or NR³R¹⁴,
wherein R¹³ is H, lower alkyl or C₃ to C₁₀ cycloalkyl, and
R¹⁴ is lower alkyl or C₃ to C₁₀ cycloalkyl, and
wherein R¹³ and R¹⁴ are independently, optionally substituted by halo, hydroxy, lower alkoxy, CN, NO₂, or optionally mono- or di-lower alkyl substituted amino;
R¹¹ is —CO—NR¹⁵R⁶, —NH—CO—R¹⁵, —CH₂—NH—C(O)—R¹⁵, —CO—R¹⁵, —S(O)—R¹⁵, —S(O)₂—R⁵, —CH₂—CO—R¹⁵ or —CH₂—NR¹⁵R¹⁶,
wherein
R¹⁵ is aryl, aryl-lower alkyl, C₃-C₁₀cycloalkyl, C₃-C₁₀cycloalkyl-lower alkyl, heterocyclyl or heterocyclyl-lower alkyl,
R¹⁶ is H, aryl, aryl-lower alkyl, aryl-lower-alkenyl, C₃-C₁₀cycloalkyl, C₃-C₁₀cycloalkyl-lower alkyl, heterocyclyl or heterocyclyl-lower alkyl, or
wherein R¹⁵ and R¹⁶ together with the nitrogen atom to which they attached are joined to form an N-heterocyclyl group,
wherein N-heterocyclyl denotes a saturated, partially unsaturated or aromatic nitrogen containing heterocyclic moiety attached via a nitrogen atom thereof having from 3 to 8 ring atoms optionally containing a further 1, 2 or 3 heteroatoms selected from N, NR¹⁷, O, S, S(O) or S(O)₂ wherein R¹⁷ is H or optionally substituted (lower alkyl, carboxy, acyl (including both lower alkyl acyl, e.g. formyl, acetyl or propionyl, or aryl acyl, e.g. benzoyl), amido, aryl, S(O) or S(O)₂), and wherein the N-heterocyclyl is optionally fused in a bicyclic structure, e.g. with a benzene or pyridine ring, and wherein the N-heterocyclyl is optionally linked in a spiro structure with a 3 to 8 membered cycloalkyl or heterocyclic ring wherein the heterocyclic ring has from 3 to 10 ring members and contains from 1 to 3 heteroatoms selected from N, NR¹⁶, O, S, S(O) or S(O)₂ wherein R¹⁶ is as defined above), and
wherein heterocyclyl denotes a ring having from 3 to 10 ring members and containing from 1 to 3 heteroatoms selected from N, NR¹⁷, O, S, S(O) or S(O)₂ wherein R¹⁷ is as defined above), and
wherein R¹⁵ and R¹⁶ are independently, optionally substituted by one or more groups, e.g. 1-3 groups, selected from halo, hydroxy, oxo, lower alkoxy, CN or NO₂, or optionally substituted (optionally mono- or di-lower alkyl substituted amino, lower-alkoxy, aryl, aryl-lower alkyl, N-heterocyclyl or N-heterocyclyl-lower alkyl (wherein the optional substitution comprises from 1 to 3 substituents selected from halo, hydroxy, lower alkoxy, lower alkoxy-lower alkyl, lower alkoxy-carbonyl, CN, NO₂, N-heterocyclyl or N-heterocyclyl-lower alkyl, or optionally mono- or di-lower alkyl substituted amino;
R¹² is is independently H, or optionally substituted (lower alkyl, aryl, aryl-lower alkyl, C₃-C₁₀cycloalkyl, C₃-C₁₀cycloalkyl-lower alkyl, heterocyclyl or heterocyclyl-lower alkyl), and
wherein R2 is optionally substituted by halo, hydroxy, oxo, lower alkoxy, CN, NO₂, or optionally mono- or di-lower alkyl substituted amino.
for simultaneous, sequential or separate use.

2. The pharmaceutical preparation according to claim 1 wherein its use is for the treatment of malignant diseases, bone metastasis, cancer cell growth, or/and cancer therapy-induced bone loss.

3. A method of treating a patient suffering from a malignant disease, bone metastasis, cancer cell growth, or/and cancer-therapy-induced bone loss comprising administering to the patient an effective amount of the pharmaceutical preparation according to claim 1.

4. A method of treating a patient suffering from a benign disease, bone loss disease, osteoporosis, osteoarthritis comprising administering to the patient an effective amount of the pharmaceutical preparation according to claim 1.

5. A pharmaceutical composition comprising zoledronic acid and a cathepsin K inhibitor for the inhibition of bone metastasis, cancer cell growth or/and inhibition of cancer-therapy-induced bone loss.

6. A pharmaceutical preparation according to claim 1, in which the cathepsin K inhibitor is selected from the group of N-[1-(Cyanomethyl-carbamoyl)-cyclohexyl]-4-(piperazin-1-yl)-benzamide; N-[1-(Cyanomethyl-carbamoyl)-cyclohexyl]-4-(4-methyl-piperazin-1-yl)-benzamide; N-[1-(Cyanomethyl-carbamoyl)-cyclohexyl]-4-(4-ethyl-piperazin-1-yl)-benzamide; N-[1-(Cyanomethyl-carbamoyl)-cyclohexyl]-4-[4-(1-propyl)-piperazin-1-yl]-benzamide; N-[1-(Cyanomethyl-carbamoyl)-cyclohexyl]-4-(4-isopropyl-piperazin-1-yl)-benzamide; N-[1-(Cyanomethyl-carbamoyl)-cyclohexyl]-4-(4-benzyl-piperazin-1-yl)-benzamide; N-[1-(Cyanomethyl-carbamoyl)-cyclohexyl]-4-[4-(2-methoxy-ethyl)-piperazin-1-yl]-benzamide; N-[1-(Cyanomethyl-carbamoyl)-cyclohexyl]-4-(1-propyl-piperidin-4-yl)-benzamide; N-[1-(Cyanomethyl-carbamoyl)-cyclohexyl]-4-[1-(2-methoxy-ethyl)-piperidin-4-yl]-benzamide; N-[1-(Cyanomethyl-carbamoyl)-cyclohexyl]-4-(1-isopropyl-piperidin-4-yl)-benzamide; N-[1-(Cyanomethyl-carbamoyl)-cyclohexyl]-4-(1-cyclopentyl-piperidin-4-yl)-benzamide; N-[1-(Cyanomethyl-carbamoyl)-cyclohexyl]-4-(1-methyl-piperidin-4-yl)-benzamide, and N-[1-(Cyanomethyl-carbamoyl)-cyclohexyl]-4-(piperidin-4-yl)-benzamide, N-[2-Cyano-4-(2,2-dimethyl-propylamino)-pyrimidin-5-ylmethyl]-4-(1-propyl-piperidin-4-yl)-benzamide, N-[2-Cyano-4-(2,2-dimethyl-propylamino)-pyrimidin-5-ylmethyl]-4-(4-methyl-piperazin-1-yl)-benzamide, N-[2-Cyano-4-(2,2-dimethyl-propylamino)-pyrimidin-5-ylmethyl]-4-[1-(2-methoxy-ethyl)-piperidin-4-yl]-benzamide, N-[2-Cyano-4-(2,2-dimethyl-propylamino)-pyrimidin-5-ylmethyl]-4-(4-propyl-piperazin-1-yl)-benzamide, N-[2-Cyano-4-(2,2-dimethyl-propylamino)-pyrimidin-5-ylmethyl]-2,2-dimethyl-3-[4-(4-methyl-piperazin-1-yl)-phenyl]-propionamide, N-[2-Cyano-4-(2,2-dimethyl-propylamino)-pyrimidin-5-ylmethyl]-2,2-dimethyl-3-[3-(4-methyl-piperazin-1-yl)-phenyl]-propionamide, N-[2-Cyano-4-(2,2-dimethyl-propylamino)-pyrimidin-5-ylmethyl]-4-(4-ethyl-piperazin-1-yl)-benzamide, N-[2-Cyano-4-(2,2-dimethyl-propylamino)-pyrimidin-5-ylmethyl]-4-(4-isopropyl-piperazin-1-yl)-benzamide, N-[2-Cyano-4-(2,2-dimethyl-propylamino)-pyrimidin-5-ylmethyl]-4-[4-(2-ethoxy-ethyl)-piperazin-1-yl]-benzamide, N-[2-Cyano-4-(2,2-dimethyl-propylamino)-pyrimidin-5-ylmethyl]-4-[4-(2-methoxy-ethyl)-piperazin-1-yl]-benzamide, N-[2-Cyano-4-(2,2-dimethyl-propylamino)-pyrimidin-5-ylmethyl]-4-piperazin-1-yl-benzamide, 4-(4-{([2-Cyano-4-(2,2-dimethyl-propylamino)-pyrimidin-5-ylmethyl]-carbamoyl}-phenyl)-piperazine-1-carboxylic acid tert-butyl ester, 4-(3-{[2-Cyano-4-(2,2-dimethyl-propylamino)-pyrimidin-5-ylmethyl]-carbamoyl}-phenyl)-piperazine-1-carboxylic acid tert-butyl ester, N-[2-Cyano-4-(2,2-dimethyl-propylamino)-pyrimidin-5-ylmethyl]-3-(4-methyl-piperazin-1-yl)-benzamide, N-[2-Cyano-4-(2,2-dimethyl-propylamino)-pyrimidin-5-ylmethyl]-3-(4-ethyl-piperazin-1-yl)-benzamide, N-[2-Cyano-4-(2,2-dimethyl-propylamino)-pyrimidin-5-ylmethyl]-3-(4-isopropyl-piperazin-1-yl)-benzamide, N-[2-Cyano-4-(2,2-dimethyl-propylamino)-pyrimidin-5-ylmethyl]-3-[4-(2-methoxy-ethyl)-piperazin-1-yl]-benzamide, N-[2-Cyano-4-(2,2-dimethyl-propylamino)-pyrimidin-5-ylmethyl]-3-[4-(2-ethoxy-ethyl)-piperazin-1-yl]-benzamide, N-[2-Cyano-4-(2,2-dimethyl-propylamino)-pyrimidin-5-ylmethyl]-4-methoxy-3-(2-pyrrolidin-1-yl-ethoxy)-benzamide, N-[2-Cyano-4-(2,2-dimethyl-propylamino)-pyrimidin-5-ylmethyl]-3-(2-dimethylamino-ethoxy)-4-methoxy-benzamide, N-[2-Cyano-4-(2,2-dimethyl-propylamino)-pyrimidin-5-ylmethyl]-4-dimethylaminomethyl-benzamide, N-[2-Cyano-4-(2,2-dimethyl-propylamino)-pyrimidin-5-ylmethyl]-4-(4-methyl-piperazin-1-ylmethyl)-benzamide, N-[2-Cyano-4-(2,2-dimethyl-propylamino)-pyrimidin-5-ylmethyl]-4-[1-(2-methoxy-ethyl)-piperidin-4-ylmethyl]-benzamide, N-[2-Cyano-4-(2,2-dimethyl-propylamino)-pyrimidin-5-ylmethyl]-4-methoxy-3-(2-piperidin-1-yl-ethoxy)-benzamide, N-[2-Cyano-4-(2,2-dimethyl-propylamino)-pyrimidin-5-ylmethyl]-3-[4-(4-ethyl-piperazin-1-yl)-phenyl]-2,2-dimethyl-propionamide or pharmaceutically acceptable salt thereof.

7. A pharmaceutical preparation according to claim 1, in which the cat K inhibitor is N-[1-(cyanomethyl-carbamoyl)-cyclohexyl]-4-(4-(1-propyl)-piperazin-1-yl)-benzamide or a pharmaceutically acceptable salt thereof and the bisphosphonate is 2-(imidazol-1yl)-1-hydroxyethane-1,1-diphosphonic acid (zoledronic acid) or pharmacologically acceptable salts thereof.
